(19) United States
(12) Patent Application Publication
(10) Pub. No.: US 2022/0144883 A1
(43) Pub. Date: May 12, 2022

(54) METHODS OF MODIFYING PHOSPHORYLATED OR SULFATED TYROSINE RESIDUES OF POLYPEPTIDES

(71) Applicant: PHOSFISH LLC, Houston, TX (US)

(72) Inventors: Carlos Augusto Gartner, Gorham, ME (US); Patrick Joseph Grimes, Houston, TX (US)

(21) Appl. No.: 17/551,971

(22) Filed: Dec. 15, 2021

Related U.S. Application Data
(63) Continuation of application No. PCT/US2021/059025, filed on Nov. 11, 2021.
(60) Provisional application No. 63/112,718, filed on Nov. 12, 2020.

(51) Int. Cl. C07K 1/00

(52) U.S. Cl. CPC C07K 1/006

(57) ABSTRACT

The present disclosure relates to methods of modifying phosphorylated or sulfated tyrosine residues of polypeptides or proteins. Benefits of the methods disclosed herein can include the specific modification of phosphorylated or sulfated tyrosine residues, and the identification, characterization and enrichment of tyrosine phosphorylated or sulfated peptides or proteins in complex biological mixtures.

### CROSS-REFERENCE

This application is a Continuation of International Application No. PCT/US21/59025, international filing date of Nov. 11, 2021, which claims priority and the benefit of U.S. Provisional Application No. 63/112,718, filed Nov. 12, 2020, both of which are incorporated in their entirety herein by reference.

### TECHNICAL FIELD

The present disclosure relates to methods of modifying phosphorylated or sulfated tyrosine residues of polypeptides or proteins. Benefits of the methods disclosed herein can include the selective modification of phosphorylated or sulfated tyrosine residues, and the identification, characterization and enrichment of tyrosine phosphorylated or sulfated peptides or proteins in complex biological mixtures.

### BACKGROUND

Analysis of the identity, quantity and activity of proteins is central to an understanding of biological processes. Among protein post-translational modifications, characterization of the phosphorylation and sulfonation of tyrosine amino acid residues is of particular interest. However, the low abundance of these residues in cells and tissues presents challenges for their detection and isolation. Several enrichment methods are currently used to select for phosphorylated polypeptides in biological samples, but these technologies lack specificity for enrichment of phosphorylated or sulfated tyrosine residues. There remains a need in the art for effective methods of selectively capturing and purifying polypeptides having phosphorylated or sulfated tyrosine residues.

### SUMMARY

The present disclosure relates to methods of modifying one or more phosphorylated or sulfated tyrosine residues of a polypeptide. In various embodiments, such a method includes modifying a polypeptide, including providing a polypeptide having at least one phosphorylated or sulfated tyrosine residue; and forming a modified polypeptide by reacting the at least one phosphorylated or sulfated tyrosine residue with an organoboronic acid, a terminal alkyne, or a terminal alkene in the presence of an amount of a transition metal catalyst, wherein the organoboronic acid, the terminal alkyne, or the terminal alkene is tethered to a structure, and wherein the transition metal catalyst contains Pd, Ni, Zn, Ir, Ru, Fe, Co, Cu, or Au, or a combination thereof. In certain embodiments, the polypeptide is a protein. In certain embodiments, the structure includes a protein, a stable isotope label, biotin, streptavidin, a dendrimer, a fluorophore, or a radioactive label.

Certain embodiments of methods herein include providing cells from a tissue or culture; and disrupting the cells to provide the polypeptide having at least one phosphorylated or sulfated tyrosine residue.

In certain embodiments, the method includes forming a molecule of Formula (I):

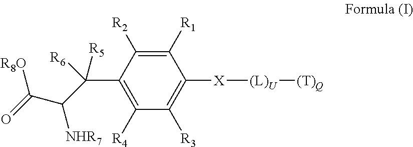

by reacting a molecule of Formula (II) or Formula (IV):

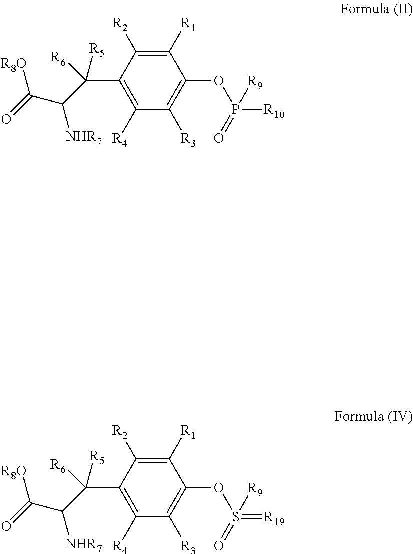

with a molecule of Formula (III):

Z-(L)_{U}-(T)_{Q}, Formula (III)

wherein X is selected from the group consisting of

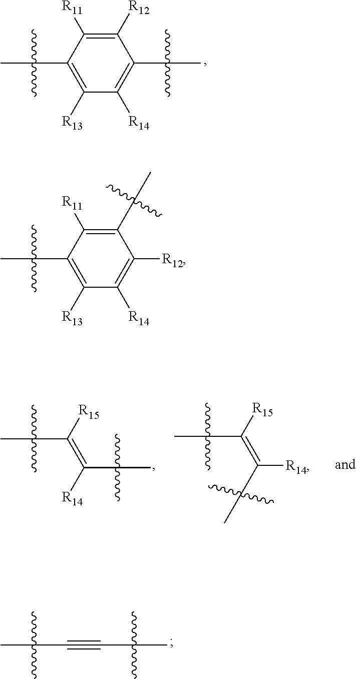

Z is selected from the group consisting of

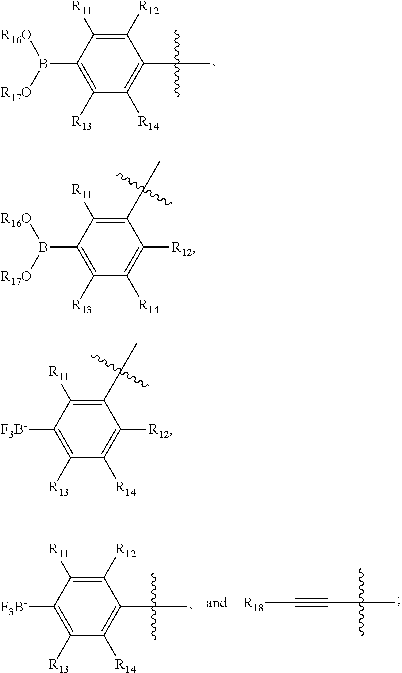

L is a linker; U is an integer of 0 or 1; T is the structure; Q is an integer of 1, 2, or 3; R₁, R₂, R₃, R₄, R₅, and R₆ are each independently selected from the group consisting of H, D, F, and C₁-C₈ alkyl; R₇ and R₈ are each independently selected from the group consisting of H, D, C₁-C₈ alkyl and a polypeptide, wherein at least one of R₇ or R₈ is a polypeptide having from 2 to about 30,000 amino acids or wherein R₇ and R₈ combine to form a polypeptide having from 2 to about 30,000 amino acids; R₉ and R₁₀ are each independently selected from the group consisting of OH, OD, ONa, OLi, OK, O—C₁-C₈ alkyl, NH—CF₃, and NH—CH₂—CF₃, R₁₁, R₁₂, R₁₃, and R₁₄ are each independently selected from the group consisting of H, D, F, and C₁-C₈ alkyl; R₁₅ is H or D; R₁₆ and R₁₇ are each independently selected from the group consisting of H, D, C₁-C₈ alkyl, Ca, Mg, Na, Li, and K; R₁₈ is H or D; and R₁₉ is selected from the group consisting of OH, OD, ONa, OLi, OK, O—C₁-C₈ alkyl, NH—CF₃, and NH—CH₂—CF₃.

In certain embodiments, the molecule of Formula (I) is selected from the group consisting of

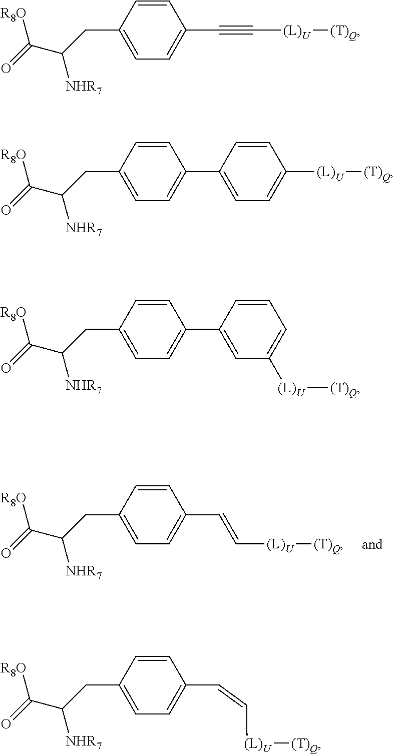

wherein R7 and R8 are each independently selected from the group consisting of H, D, C1-C8 alkyl and a polypeptide, wherein at least one of R7 or R8 is a polypeptide having from 2 to about 30,000 amino acids or wherein R7 and R8 combine to form a polypeptide having from 2 to about 30,000 amino acids; L is a linker; U is an integer of 0 or 1; T is the structure; and Q is an integer of 1, 2, or 3. In certain embodiments, the R₇ and R₈ bridge to form a protein.

In certain embodiments, the method includes forming a molecule for Formula (I) by reacting the molecule of Formula (II) with the molecule of Formula (III).

In certain embodiments, the method includes forming a molecule for Formula (I) by reacting the molecule of Formula (IV) with the molecule of Formula (III).

In some embodiments of methods herein, a modified polypeptide is formed by reacting a phosphorylated tyrosine residue with an organoboronic acid or a terminal alkyne. In other embodiments, a modified polypeptide is formed by reacting a sulfated tyrosine residue with an organoboronic acid or a terminal alkyne.

In certain embodiments, the transition metal catalyst includes a dialkyl biaryl-ligated palladium complex, a carbene-ligated palladium complex, [1,3-Bis(2,6-Diisopropylphenyl)imidazol-2-ylidene](3-chloropyridyl)palladium(II) dichloride; Dichloro[1,3-bis(2,6-Di-3-pentylphenyl)imidazol-2-ylidene](3-chloropyridyl)palladium(II); (1,3-Bis(2,6-diisopropylphenyl)imidazolidene) (3-chloropyridyl) palladium(II) dichloride; or a compound of Formula (IV)

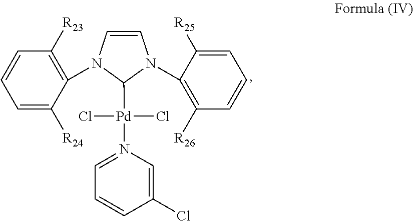

wherein R₂₃, R₂₄, R₂₅, and R₂₆ are each independently selected from a methyl group, an ethyl group, an isopropyl group, an isopentyl group, and an isoheptyl group, or a salt or mixture thereof.

In certain embodiments, the transition metal catalyst includes palladium ligated to a ligand, wherein the ligand includes Sodium 2′-dicyclohexylphosphino-2,6-dimethoxy-1,1′-biphenyl-3-sulfonate hydrate; 2-Dicyclohexylphosphino-2′,6′-diisopropoxybiphenyl; 2-Dicyclohexylphosphino-2′,4′,6′-triisopropylbiphenyl; 2′-(Diphenylphosphino)-N,N′-dimethyl-(1,1′-biphenyl)-2-amine, 2-Diphenylphosphino-2′-(N,N-dimethylamino)biphenyl; 2-Dicyclohexylphosphino-2′-methylbiphenyl, 2-Methyl-2′-dicyclohexylphosphinobiphenyl; (2-Biphenyl)di-tert-butylphosphine; 2′-Dicyclohexylphosphino-2,4,6-trimethoxybiphenyl; 2′-Dicyclohexylphosphino-2-methoxy-1-phenylnaphthalene; 2-Dicyclohexylphosphino-2′-(N,N-dimethylamino)biphenyl; (2-Biphenyl)dicyclohexylphosphine, 2-(Dicyclohexylphosphino)biphenyl; 2-(Dicyclohexylphosphino)3,6-dimethoxy-2′,4′,6′-triisopropyl-1,1′-biphenyl; or a combination or mixture thereof.

In certain embodiments, the linker includes a polyethylene glycol segment or has a formula: —NH((CH₂)₂O)ₙ(CH₂)₂NH—, where n is an integer of from 0 to 50, or wherein the dendrimer includes —(CH₂)₃— or —CH₂—CONH—CH₂— dendrons.

Certain methods of modifying a polypeptide herein include forming a molecule of Formula (I):

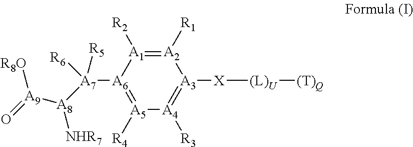

by reacting a molecule of Formula (II) or a molecule of Formula (IV):

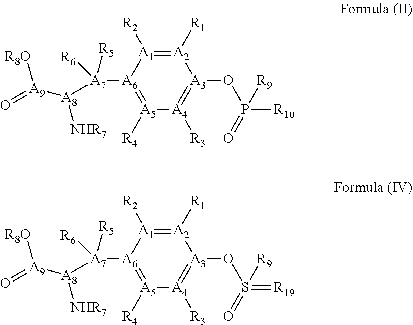

with a molecule of Formula (III):

Z-(L)_{U}-(T)_{Q}, Formula (III)

wherein X is selected from the group consisting of

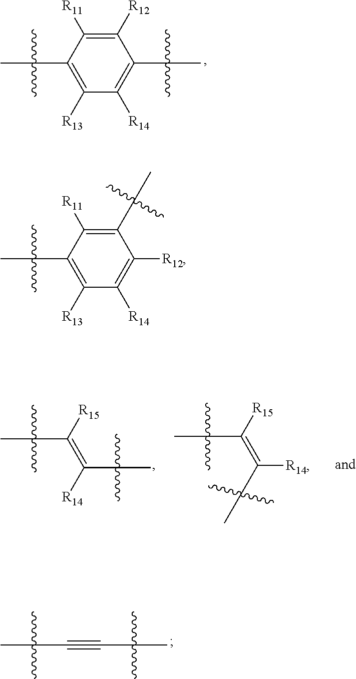

Z is selected from the group consisting of

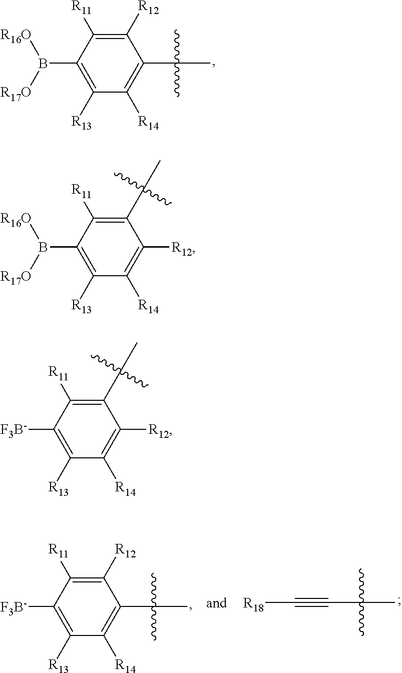

Y is selected from the group consisting of P and S; L is a linker; U is an integer of 0 or 1; T is the structure; Q is an integer of 1, 2, or 3; R₁, R₂, R₃, R₄, R₅, and R₆ are each independently selected from the group consisting of H, D, F, ¹²C₁-¹²C₈ alkyl, and ¹³C₁-¹³C₈; R₇ and R₈ are each independently selected from the group consisting of H, D, ¹³C₁-¹³C₈, C₁-C₈ alkyl and a polypeptide, wherein at least one of R₇ or R₈ is a polypeptide having from 2 to about 30,000 amino acids or wherein R₇ and R₈ combine to form a polypeptide having from 2 to about 30,000 amino acids; R₉ and R₁₀ are each independently selected from the group consisting of OH, OD, ONa, OLi, OK, O—C₁-C₈ alkyl, NH—CF₃, and NH—CH₂—CF₃, R₁₁, R₁₂, R₁₃, and R₁₄ are each independently selected from the group consisting of H, D, F, and C₁-C₈ alkyl; R₁₅ is H or D; R₁₆ and R₁₇ are each independently selected from the group consisting of H, D, C₁-C₈ alkyl, Ca, Mg, Na, Li, and K; R₁₈ is H or D, R₁₉ is selected from the group consisting of OH, OD, ONa, OLi, OK, O—C₁-C₈ alkyl, NH—CF₃, and NH—CH₂—CF₃, and A₁, A₂, A₃, A₄, A₅, A₆, A₇, A₈, and A₉ are each independently selected from the group consisting of C¹² and C¹³.

### DETAILED DESCRIPTION

Unless otherwise noted, all measurements are in standard metric units.

Unless otherwise noted, all instances of the words “a,” “an,” or “the” can refer to one or more than one of the word that they modify.

Unless otherwise noted, the phrase “at least one of” means one or more than one of an object. For example, “at least one phosphorylated or sulfated tyrosine residue” means a single phosphorylated tyrosine residue, a single sulfated tyrosine residue, more than one phosphorylated tyrosine residue, more than one sulfated tyrosine residue, or any combination thereof.

Unless otherwise noted, the term “about” refers to ±10% of the non-percentage number that is described, rounded to the nearest whole integer. For example, about 30,000 amino acids, would include 27,000 amino acids to 33,000 amino acids. Unless otherwise noted, the term “about” refers to ±5% of a percentage number. For example, about 20% would include 15 to 25%. When the term “about” is discussed in terms of a range, then the term refers to the appropriate amount less than the lower limit and more than the upper limit. For example, from 2 to about 30,000 amino acids would include from 2 to 33,000 amino acids.

Unless otherwise noted, the term “structure” refers to a molecule, a compound, or a surface of a solid material. For example, a structure might include a surface of a solid bead used for immobilization of proteins, such a SEPHAROSE® bead, or a silicon or silicon oxide surface for lab-on-a-chip applications.

Unless otherwise noted, a reference to average molecular weight refers to a number average molecular weight.

Several technologies are available for the capture and labeling of phosphate-containing polypeptides. One technology that is commonly used to enrich for phosphoproteins in biological samples is the immobilized metal ion affinity chromatography (IMAC) method. This method uses immobilized transition metals such as iron, titanium, and zirconium; the affinity of the negatively charged phosphate groups for the positively charged metal ions allows phosphorylated peptides to be enriched from peptide samples. This method captures all phosphorylated peptides and proteins, including those phosphorylated on serine and threonine residues as well as tyrosine. Selectivity of the IMAC method is also limited when working with complex samples, especially whole cell extracts, because of contamination from nonspecific binding of non-phosphorylated and often highly acidic peptides, which can also have a high binding affinity for the metal ions. Other technologies make use of metal oxide chelation to enrich for phosphopeptides in the profiling of phosphoproteomes. Titanium dioxide metal oxide affinity chromatography (TiO₂-MOAC) is widely regarded as more selective than IMAC for phosphopeptide enrichment. However, like IMAC, it captures all phosphorylated peptides and proteins, and does not have specificity or selectivity for polypeptides or proteins phosphorylated on tyrosine residues. The Strong Cation Exchange (SCX) method is a widely used HPLC fractionation method that separates peptides by solution charge. The SCX stationary phase typically uses negatively charged aliphatic sulfonic acid groups. The presence of phosphate groups in polypeptides adds a negative charge at pH 2.7, at which pH phosphopeptides are expected to elute earlier than their non-phosphorylated homologs. This method also lacks specificity for phosphorylated tyrosine residues. Antibody-mediated immunoprecipitation can be selective for tyrosine-phosphorylated peptides and proteins by using an anti-pTyr antibody. However, results of studies employing this technology have often been reported to be unsatisfactory. The antibody specificity tends to be overly sensitive to the peptide sequence of the antigen used to generate the antibody, rather than being specific to the phosphotyrosine entity alone.

Adding to the challenges of the capture and labeling of tyrosine-phosphorylated peptides is their relatively low abundance in cells and tissues. Tyrosine phosphorylation accounts for less than ten percent of phosphorylation sites in proteins, so that their identification and characterization are often masked by readouts from serine- and threonine-phosphorylated entities. Tyrosine sulfation is also of interest in biochemistry and proteomics. Tyrosine sulfation can be distinguished from tyrosine phosphorylation by means of a strict consensus amino acid sequence that is specific for tyrosine sulfation sites.

Transition metal catalyzed cross-coupling reactions have become widely used in industrial and research synthetic chemistry as a methodology for forming C—C and C-Heteroatom bonds. Generally, these reactions join two organic groups together with the assistance of a transition metal catalyst. Generally, an organometallic compound of the type R-M reacts with an organohalide of the type R′-M to form a new C—C bond between the R and R′ groups. Palladium catalysts are prominently used in these reactions, although other transition metal catalysts can be effective. These reactions are believed to proceed via a catalytic cycle of oxidative addition of the organohalide substrate to the transition metal catalyst to form an intermediate, followed by transmetallation in which the organometallic cross-coupling reagent reacts with the intermediate to replace the halide with the R′ group, followed by rearrangement and reductive elimination to form the R—R′ bond and regenerate the catalyst. The transition metal catalyst is typically selected to have properties that will facilitate the desired reaction. Catalysts are often selected to have strong σ-donating ligands, such as trialkylphosphines, to increase electron density around the metal and accelerate the oxidative addition of the catalyst to the substrate, which is believed to be the rate-limiting step. The elimination step is also accelerated by bulky ligands, especially phosphine ligands having a large Tolman cone angle, which can destabilize the rearranged complex. The transmetallation step is favored by R′ being an electron rich group, and by a lack of steric hindrance on R and R′.

Several types of cross-coupling reactions have been developed based on the use of different substrates. The Suzuki coupling reaction involves the cross-coupling of an organohalide substrate (or its equivalent) with an organoboron cross-coupling reagent, in the presence of a suitable transition metal catalyst. The organoboron reagent, often in the form of an organoboronic acid or ester, may require activation by a base or fluoride to enable it to undergo transmetallation. The Suzuki coupling reaction is commonly used to form aryl-aryl bonds, although the reaction is highly tolerant of a wide variety of different functional groups. Another type of coupling is the Negishi reaction, which uses organozinc reagents to couple with organohalides and their equivalents, in the presence of a transition metal catalyst. The Negishi coupling is compatible with a range of organohalide functional groups, including ketones, esters, amines and nitriles. Another type of coupling reaction is the Sonogashira coupling, which typically couples an alkyne group with an organohalide or its equivalent, and is especially useful for the formation of aryl- and alkenyl-alkynes.

It has been discovered that such coupling reactions can provide specifically react with a phosphorylated or sulfated tyrosine to selectively modify a polypeptide or protein. In more detail, embodiments of the present disclosure can provide methods of modifying a polypeptide having at least one phosphorylated or sulfated tyrosine residue, by reacting at least one phosphorylated or sulfated tyrosine residue as a substrate in a cross-coupling reaction with a cross-coupling reagent, in the presence of a transition metal catalyst. It has also been discovered that the methods disclosed herein can provide a modified polypeptide by selecting a transition metal catalyst and a cross-coupling reagent that can react with the phosphorylated or sulfated tyrosine residue in a specific manner. It is believed that the reason for this specificity is that phosphorylated or sulfated residues of threonine and serine are inert to the cross-coupling reactions of the embodied methods. Thus, the embodied methods herein can provide a benefit of polypeptide modification that is specific for modification of phosphorylated or sulfated tyrosine residues.

Embodiments herein can provide for the tethering of a label or a purification handle or other structure specifically to a phosphorylated or sulfated tyrosine residue in a polypeptide, using the cross-coupling reactions of the embodied methods. Such embodiments can provide a benefit of visualizing or detecting a polypeptide having at least one phosphorylated or sulfated tyrosine residue in a cell or tissue culture, including such polypeptide targets having a very low abundance in complex cellular or tissue samples. Embodiments herein can provide a benefit of enriching and purifying tyrosine-phosphorylated or tyrosine-sulfated polypeptide targets of very low abundance from a complex mixture of non-tyrosine phosphorylated or non-tyrosine sulfated polypeptides. Such embodiments can provide a benefit of facilitating the manipulation and characterization of such low abundance target polypeptides.

The significance of the embodied methods and their potential impact on research cannot be overstated. Proteins that are critical to disease states, including cancer, have often been found to be present at a very low copy number in cells, making them very difficult to detect in complex biological samples. Through providing methods that can specifically modify, label or capture polypeptides having phosphorylated or sulfated tyrosine residues, embodiments of methods herein can provide a benefit of detecting, capturing, and characterizing low abundance target proteins having significance for the progress of research and drug development related to disease.

### Embodiments of Methods of Modifying a Polypeptide

Embodied methods of modifying a polypeptide as disclosed herein can include providing a polypeptide having at least one phosphorylated or sulfated tyrosine residue. In certain embodiments, the polypeptide can be a protein. In certain embodiments, the at least one phosphorylated or sulfated tyrosine residue can be located at the N-terminus, at the C-terminus, or at an internal location in the polypeptide's amino acid sequence. In various embodiments, a modified polypeptide can be formed by reacting the at least one phosphorylated or sulfated tyrosine residue with an organoboronic acid, a terminal alkyne, or a terminal alkene in the presence of an amount of a transition metal catalyst. In certain embodiments, the modified polypeptide is formed by reacting a phosphorylated tyrosine residue with the organoboronic acid or the terminal alkyne. In certain embodiments, the modified polypeptide is formed by reacting a sulfated tyrosine residue with the organoboronic acid or the terminal alkyne. In various embodiments, the organoboronic acid, the terminal alkyne, or the terminal alkene is tethered to a structure. In certain embodiments, the structure includes a protein, a stable isotope label, biotin, streptavidin, a dendrimer, a fluorophore, or a radioactive label. In certain embodiments, the transition metal catalyst contains Pd, Ni, Zn, Ir, Ru, Fe, Co, Cu, or Au, or a combination thereof.

In certain embodiments, the at least one phosphorylated or sulfated tyrosine residue can be reacted with an organoboronic acid, a terminal alkyne, or a terminal alkene in the presence of an amount of a transition metal catalyst, and in the presence of a base reagent or an acid reagent. A base reagent can include a mild bio-compatible base, such as a bicarbonate, borate, or ammonia base. In certain embodiments, a base reagent can include a non-nucleophilic hindered base, such as diazabicycloundecene (DBU), diisopropylethylamine (DIEA), and 2,6-lutidine. An acid reagent can include a Lewis acid such as aluminum, magnesium, zinc, or trivalent boron.

Cross-coupling reactions of embodied methods can be conducted at room temperature. In certain embodiments, the cross-coupling reactions can be conducted at a temperature of up to about 80 degrees Celsius for a time period of about one hour or less. Cross-coupling reactions of various embodied methods may be conducted at atmospheric pressure. Cross-coupling reactions of various embodiments can be conducted using a solvent that can dissolve polypeptides and proteins. In certain embodiments, the solvent can include a polar solvent. Certain embodiments can be carried out including an aqueous solvent. A solvent can be selected in various embodiments according to catalyst stability. In certain embodiments, cross-coupling reactions can be conducted in an alcoholic solvent, or in a polar aprotic solvent, such as dimethylsulfoxide (DMSO) or N,N-dimethylformamide (N,N-DMF).

Certain embodiments of methods herein include providing cells from a tissue or culture, and disrupting the cells to provide a polypeptide having at least one phosphorylated or sulfated tyrosine residue. Such a polypeptide can be modified according to embodiments of the methods herein. In certain embodiments, cells may be harvested from a tissue or from a cell culture by a suitable method. One such cell culture harvesting method can include scraping cells from a plate-grown culture in the presence of a denaturing buffer. Another such harvesting method can include centrifugation and homogenization of cells grown in a suspension culture. Harvesting methods that can be suitable for providing cells from a tissue sample can include disruption, such as by sonication, followed by homogenization, such as by use of a French press or pestle and barrel homogenizer, or a combination of suitable techniques.

In certain embodiments of methods herein, the at least one phosphorylated or sulfated tyrosine residue can be reacted with an organoboronic acid, a terminal alkyne, or a terminal alkene in the presence of an amount of transition metal catalyst, without activation of the phosphate group or sulfonate group of the at least one phosphorylated or sulfated tyrosine residue prior to reaction with the cross-coupling reagent. Certain other embodiments of methods herein can include activating the phosphate or sulfonate group of the at least one phosphorylated or sulfated tyrosine residue prior to reaction with a cross-coupling reagent. In one such embodiment, the phosphate group of at least one phosphorylated tyrosine residue can be activated by reaction with the diazo form of 2,2,2-trifluoroethylamine, or with the diazo form of 2,2,2-trichloroethylamine.

### Transition Metal Catalysts of Various Embodiments

In various embodied methods, a modified polypeptide can be formed by reacting at least one phosphorylated or sulfated tyrosine residue in the polypeptide sequence with an organoboronic acid, a terminal alkyne, or a terminal alkene in the presence of an amount of a transition metal catalyst. A catalyst suitable for use in the cross-coupling reactions of various embodied methods can be selected to form an oxidative addition intermediate with the at least one phosphorylated or sulfated tyrosine residue of a polypeptide. In such embodiments, the phosphate or sulfate group on the aryl ring of the tyrosine residue can serve as a leaving group. In certain embodiments, a catalyst can be selected that can form a reaction intermediate without the prior activation of the phosphate or sulfonate group on the tyrosine residue. In certain embodiments, the catalyst can include a palladium catalyst in a Suzuki cross-coupling reaction. Such a palladium catalyst can include a Buchwald-Hartwig dialkylbiaryl phosphine-ligated neutral palladium complex (PHOS) palladium catalyst. In other embodiments, a palladium catalyst can include a carbene-ligated palladium complex catalyst, or a pyridine enhanced precatalyst preparation stabilization and initiation (PEPPSI) catalyst. Other embodiments can include a nickel, zinc, or iron-based catalyst. In certain embodiments, the catalyst can include palladium ligated to one or more ligands, wherein a ligand can be selected to facilitate the catalytic cycle of the cross-coupling reaction. Certain embodiments can include a strong σ-donating ligand, such as a trialkylphosphine ligand, or a bulky ligand, such as a phosphine ligand having a large Tolman cone angle.

In certain embodiments, the transition metal catalyst includes a dialkyl biaryl-ligated palladium complex, a carbene-ligated palladium complex, [1,3-Bis(2,6-Diisopropylphenyl)imidazol-2-ylidene](3-chloropyridyl)palladium(II) dichloride; Dichloro[1,3-bis(2,6-Di-3-pentylphenyl)imidazol-2-ylidene](3-chloropyridyl)palladium(II); (1,3-Bis(2,6-diisopropylphenyl)imidazolidene) (3-chloropyridyl) palladium(II) dichloride; or a compound of Formula (IV)

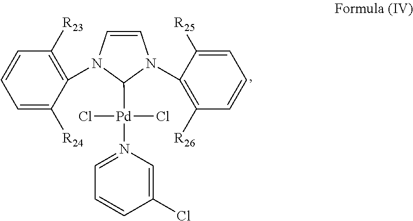

wherein R₂₃, R₂₄, R₂₅, and R₂₆ are each independently selected from a methyl group, an ethyl group, an isopropyl group, an isopentyl group, and an isoheptyl group, or a salt or mixture thereof.

In certain embodiments, the transition metal catalyst includes palladium ligated to a ligand, wherein the ligand includes Sodium 2′-dicyclohexylphosphino-2,6-dimethoxy-1,1′-biphenyl-3-sulfonate hydrate; 2-Dicyclohexylphosphino-2′,6′-diisopropoxybiphenyl; 2-Dicyclohexylphosphino-2′,4′,6′-triisopropylbiphenyl; 2′-(Diphenylphosphino)-N,N′-dimethyl-(1,1′-biphenyl)-2-amine, 2-Diphenylphosphino-2′-(N,N-dimethylamino)biphenyl; 2-Dicyclohexylphosphino-2′-methylbiphenyl, 2-Methyl-2′-dicyclohexylphosphinobiphenyl; (2-Biphenyl)di-tert-butylphosphine; 2′-Dicyclohexylphosphino-2,4,6-trimethoxybiphenyl; 2′-Dicyclohexylphosphino-2-methoxy-1-phenylnaphthalene; 2-Dicyclohexylphosphino-2′-(N,N-dimethylamino)biphenyl; (2-Biphenyl)dicyclohexylphosphine, 2-(Dicyclohexylphosphino) biphenyl; 2-(Dicyclohexylphosphino)3,6-dimethoxy-2′,4′,6′-triisopropyl-1,1′-biphenyl; or a combination or mixture thereof.

### Cross-Coupling Reagents of Various Embodiments

Cross-coupling reactions of various embodiments can result in the formation of C—C bonds between the aromatic ring of the at least one phosphorylated or sulfated tyrosine residue and an organic residue of the cross-coupling reagent. Cross-coupling reagents in various embodiments can include organoboron reagents, such as an organoboronic acid or an organoboronic ester (Suzuki coupling); an organozinc reagent (Negishi coupling); or a terminal alkyne reagent (Sonogashira coupling). The organic residue coupled with the tyrosine aromatic ring structure can include a variety of suitable groups, including but not limited to aryl, heteroaryl, alkenyl, alkynyl, alkyl, and organosilicon functional groups.

In various embodiments, a modified polypeptide is formed by reacting at least one phosphorylated or sulfated tyrosine residue with an organoboronic acid, a terminal alkyne, or a terminal alkene in the presence of an amount of a transition metal catalyst. In certain embodiments, a cross-coupling reagent includes an aryl boronic acid reagent that can be reacted with the at least one phosphorylated or sulfated tyrosine residue in a Suzuki cross-coupling reaction. In such embodiments, the aryl group can be modified with electron-withdrawing or electron-donating groups to adjust the reactivity of the aryl boronic acid reagent. In certain embodiments, a cross-coupling reagent includes a vinyl boronic acid reagent. In certain embodiments, a cross-coupling reagent includes a terminal alkyne reagent.

In various embodiments, an organoboronic acid, a terminal alkyne, or a terminal alkene cross-coupling reagent is tethered to a structure. In certain embodiments, the structure can include a protein, a stable isotope label, biotin or an analog thereof, a biorthogonal linker, streptavidin, a dendrimer, a fluorophore, or a radioactive label. In various embodiments, the organic residue to be coupled to the at least one phosphorylated or sulfated tyrosine residue can be tethered to such a structure. Structures in various embodiments can have a benefit of providing a label or tag for the detection or purification of a polypeptide modified according to embodied methods herein. For example, an amount of a modified polypeptide in a sample, including a sample from a cell culture or a tissue, can be quantified by measuring an amount of a fluorescent dye or a radioactive tracer in the sample.

Another benefit can include the purification of a modified polypeptide from a sample, including a sample from a cell culture or a tissue, via a purification “handle” incorporated into the structure. Such a handle can include a moiety to allow the attachment of a modified polypeptide to a solid phase such as a resin support. For example, a target protein containing a phosphorylated or sulfated tyrosine residue can be modified by embodied methods herein using a cross-coupling reagent tethered to a structure having a reversible binding characteristic with a ligand. The modified target protein can then be bound to a ligand that is immobilized on a chromatography resin or bead; non-specific proteins will not bind. The target protein can then be eluted from the resin or bead under conditions reversing the structure-ligand binding, thus allowing a target protein to be purified from a complex biological mixture.

In certain embodiments, the cross-coupling reagent can be tethered directly to a structure. In certain embodiments, the cross-coupling reagent can be tethered to a structure via a linker. Such a linker can selected to have suitable chemical and structural characteristics, for example, having a length suitable for the size or configuration of the polypeptide or protein to be modified. A linker can provide a benefit of facilitating the detection, purification, or further modification of a large polypeptide or a large protein in a complex biological sample. In certain embodiments, a linker can include a chemically robust and water-soluble linker, such as a polyethylene glycol (PEG)-based linker. In certain embodiments, a linker can include a cleavable site that can be cleaved under conditions that are mild with respect to biomolecules. In an aspect, the linker can be cleaved only under specific reductive conditions. A cleavable linker can provide a benefit of allowing a modified polypeptide to be released from a solid support in an identification or purification process. An additional benefit of a cleavable linker can be to facilitate further desired modifications to the polypeptide or protein.

### Cross-Coupling Reactions of Various Embodiments

Certain embodiments of methods of modifying a polypeptide herein include forming a modified polypeptide molecule of Formula (I):

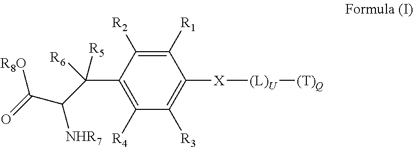

by reacting a polypeptide molecule having at least one phosphorylated of Formula (II) or sulfated tyrosine residue of Formula (IV):

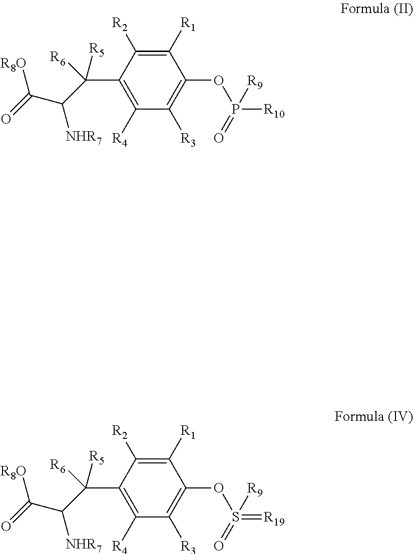

with a cross-coupling reagent molecule of Formula (III):

Z-(L)_{U}-(T)_{Q}, Formula (III)

In certain embodiments, X in the modified polypeptide of Formula I can include an organic residue having the following structures:

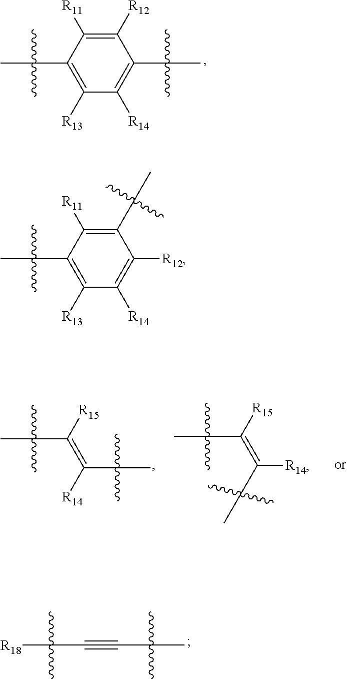

In certain embodiments, Z in the cross-coupling reagent of Formula III includes an organoboron reagent or a terminal alkyne reagent containing an organic residue to be coupled with the at least one phorphorylated or sulfated tyrosine residue, and can include the following structures:

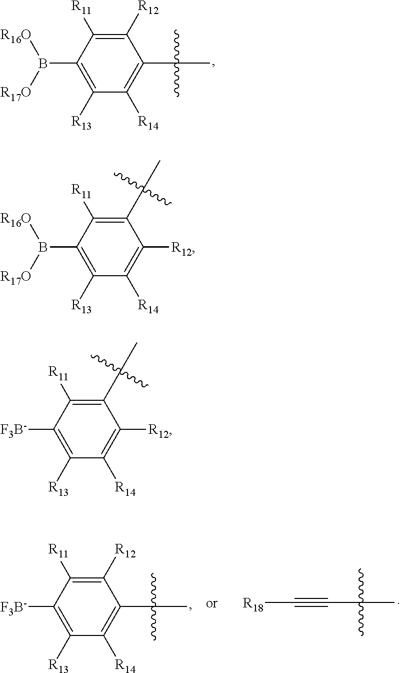

In certain embodiments, the at least one tyrosine residue can be phosphorylated or sulfated. In certain embodiments, the cross-coupling reagent can optionally include a linker; thus, L in Formula III is a linker, and U is an integer of 0 or 1. In certain embodiments, the cross-coupling reagent is tethered to one or more structures. An embodiment including Formula III can include one, two, or three structures T, where Q is an integer of 1, 2, or 3.

In certain embodiments, R₁, R₂, R₃, R₄, R₅, and R₆ in Formulas I and II can each independently include H, D, F, and C₁-C₈ alkyl; R₇ and R₈ can each independently include H, D, C₁-C₈ alkyl and a polypeptide, wherein at least one of R₇ or R₈ is a polypeptide having from 2 to about 30,000 amino acids, or wherein R₇ and R₈ combine to form a polypeptide having from 2 to about 30,000 amino acids. In such embodiments, the at least one phosphorylated of sulfated tyrosine residue can be positioned at the N-terminus, the C-terminus, or at any internal position in the amino acid sequence of the polypeptide. In certain embodiments, R₉ and R₁₀ in Formula II can each independently include OH, OD, ONa, OLi, OK, O—C₁-C₈ alkyl, NH—CF₃, and NH—CH₂—CF₃.

In certain embodiments including X in Formula I and Z in Formula III, R₁₁, R₁₂, R₁₃, and R₁₄ can each independently include H, D, F, and C₁-C₈ alkyl; R₁₅ is H or D; R₁₆ and R₁₇ can each independently include H, D, C₁-C₈ alkyl, Ca, Mg, Na, Li, and K; and R₁₈ is H or D.

In certain embodiments, the modified polypeptide of Formula (I) can include the following structures:

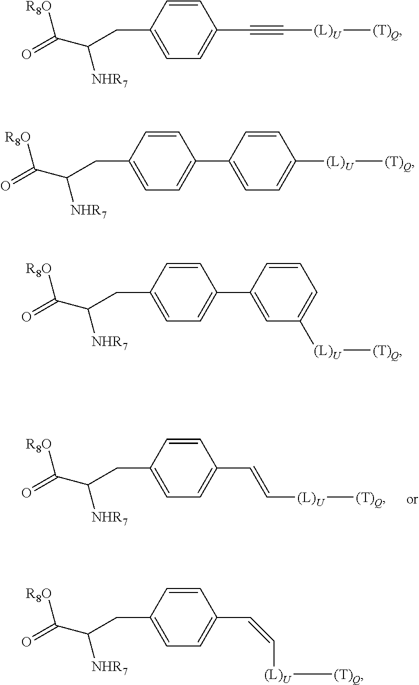

wherein R₇, R₅, L, U, T, and Q are as described above.

Certain embodiments of methods of modifying a polypeptide herein include forming a modified polypeptide molecule of Formula (I):

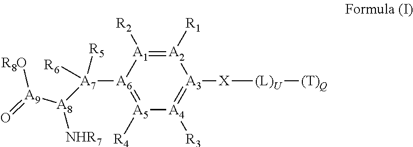

by reacting a polypeptide having at least one phosphorylated or sulfated tyrosine residue of Formula (II):

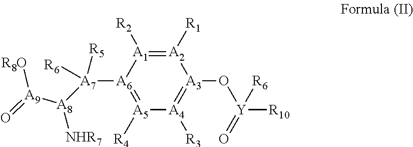

with a cross-coupling reagent molecule of Formula (III):

Z-(L)_{U}-(T)_{Q}, Formula (III)

In certain embodiments, X in the modified polypeptide of Formula I can include an organic residue having the following structures:

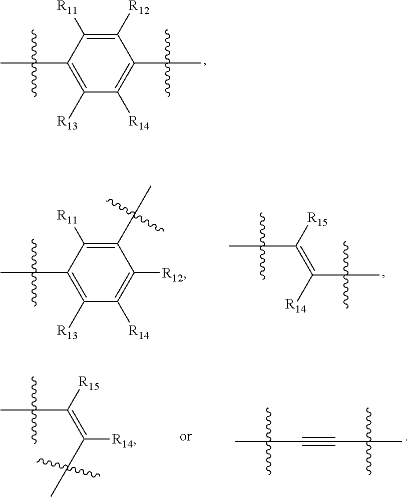

In certain embodiments, Z in the cross-coupling reagent of Formula III includes an organoboron reagent or a terminal alkyne reagent containing an organic residue to be coupled with the at least one phorphorylated or sulfated tyrosine residue, and can include the following structures:

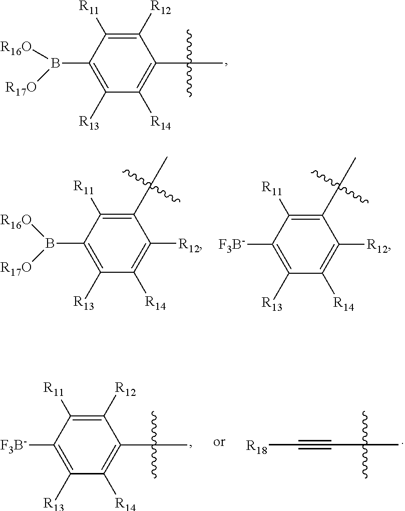

In certain embodiments, the at least one tyrosine residue can be phosphorylated or sulfated. In certain embodiments, the cross-coupling reagent can optionally include a linker; thus, L in Formula III is a linker, and U is an integer of 0 or 1. In certain embodiments, the cross-coupling reagent is tethered to one or more structures. An embodiment including Formula III can include one, two, or three structures T, where Q is an integer of 1, 2, or 3.

In certain embodiments, R₁, R₂, R₃, R₄, R₅, and R₆ in Formulas I and II can each independently include H, D, F, and C₁-C₈ alkyl; R₇ and R₈ can each independently include H, D, C₁-C₈ alkyl and a polypeptide, wherein at least one of R₇ or R₈ is a polypeptide having from 2 to about 30,000 amino acids, or wherein R₇ and R₈ combine to form a polypeptide having from 2 to about 30,000 amino acids. In such embodiments, the at least one phosphorylated of sulfated tyrosine residue can be positioned at the N-terminus, the C-terminus, or at any internal position in the amino acid sequence of the polypeptide. In certain embodiments, R₉ and R₁₀ in Formula II can each independently include OH, OD, ONa, OLi, OK, O—C₁-C₈ alkyl, NH—CF₃, and NH—CH₂—CF₃.

In certain embodiments including X in Formula I and Z in Formula III, R11, R12, R13, and R₁₄ can each independently include H, D, F, and C₁-C₈ alkyl; R₁₅ is H or D; R₁₆ and R₁₇ can each independently include H, D, C₁-C₈ alkyl, Ca, Mg, Na, Li, and K; and R₁₅ is H or D.

In certain embodiments, A₁, A₂, A₃, A₄, A₅, A₆, A₇, A₈, and A₉ in Formulas I and II are each independently selected from the group consisting of ¹²C and ¹³C. Such embodiments can provide a benefit of detecting and characterizing a polypeptide having at least one phosphorylated or sulfated tyrosine residue, through incorporation of radioactive labeling into the cross-coupling reaction product.

In certain embodiments, the method includes use of a molecule of Formula (III) to selectively modify a molecule of formula (II) or a molecule of Formula (IV) to form a molecule of Formula (I), wherein L, U, T, Q, and R₁-R₁₈ are as defined above.

A product is disclosed herein. In certain embodiments, a molecule of Formula (I) is disclosed. In certain embodiments, a product of the method of forming Formula (I) is disclosed.

### EXAMPLES

### Example 1. Model Systems Employing Small Molecules: Suzuki Cross-Coupling Reactions

The amino acid O-phosphotyrosine was used as a model system for O-phosphotyrosine-containing polypeptides for adaptation of reaction conditions for metal-catalyzed cross-coupling reactions. Use of a simple model system such as this is important in order to ensure completeness of reaction in a small molecule. The simplicity of the test molecule as a model for more complicated polypeptides facilitates adaptation of reaction conditions for larger peptides and proteins.

### Model Systems Employing Unactivated O-Phosphotyrosine and Arylboronic Acid

### Example 1.1

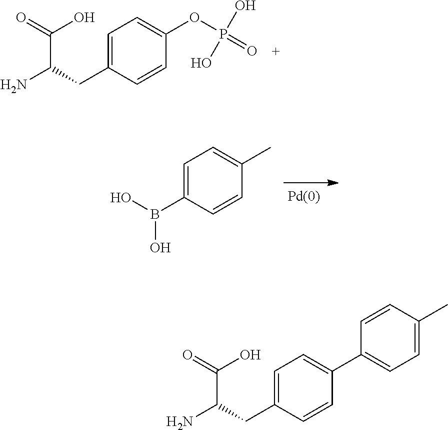

Tyrosine-O-phosphate (Tyr-O-Phos, 261 μg, 1 μmol) is dissolved in N,N-DMF containing 20% water, 10 μmol p-tolylboronic acid (1.36 mg), and potassium carbonate (K₂CO₃, 690 μg, 5 μmol). To the reaction solution is added 0.1 μmol of a PHOS palladium catalyst as a 10 μM stock solution in N,N-DMF. The reaction mixture is warmed to 50° C. with stirring. Reaction progression is monitored by liquid chromatography-mass spectrometry in which formation of the product is seen ([M+H]¹⁺=253.13 Da) with concurrent disappearance of the starting material, Tyr-O-Phos ([M+H]¹⁺=262.04 Da).

### Example 1.2

Example 1.2 is performed following the procedure of Example 1.1, except that potassium phosphate is used instead of potassium carbonate. Molar concentration of the base is maintained.

### Example 1.3

Example 1.3 is performed following the procedure of Example 1.1, except that diisopropylethylamine (DIEA) is used instead of potassium carbonate. Molar concentration of the base is maintained.

### Example 1.4

Example 1.4 is performed following the procedure of Example 1.1, except that potassium hydroxide (KOH) is used instead of potassium carbonate. Molar concentration of the base is maintained.

### Example 1.5

Example 1.5 is performed following the procedure of Example 1.1, except that the palladium catalyst is in the form of a pyridine-enhanced precatalyst preparation stabilization and initiation (PEPPSI) reagent. Molar concentration of the catalyst is maintained equal to that employed using the PHOS catalyst.

### Example 1.6

Example 1.6 is performed following the procedure of Example 1.2, except that the palladium catalyst is in the form of a pyridine-enhanced precatalyst preparation stabilization and initiation (PEPPSI) reagent. Molar concentration of the catalyst is maintained equal to that employed using the PHOS catalyst.

### Example 1.7

Example 1.7 is performed following the procedure of Example 1.3, except that the palladium catalyst is in the form of a pyridine-enhanced precatalyst preparation stabilization and initiation (PEPPSI) reagent. Molar concentration of the catalyst is maintained equal to that employed using the PHOS catalyst.

### Example 1.8

Example 1.8 is performed following the procedure of Example 1.4, except that the palladium catalyst is in the form of a pyridine-enhanced precatalyst preparation stabilization and initiation (PEPPSI) reagent. Molar concentration of the catalyst is maintained equal to that employed using the PHOS catalyst.

Model systems employing unactivated O-phosphotyrosine and potassium aryltrifluoroborate Example 1.9:

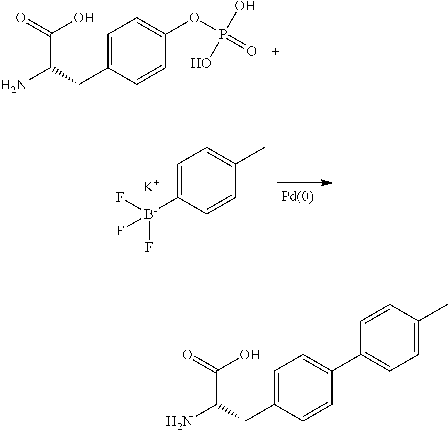

Example 1.9 is performed following the procedure of Example 1.1, except that potassium p-tolyltrifluoroborate is used instead of p-tolylboronic acid. Molar concentration of the boronic acid derivative is the same as that of the p-tolylboronic acid.

### Example 1.10

Example 1.10 is performed following the procedure of Example 1.2, except that potassium p-tolyltrifluoroborate is used instead of p-tolylboronic acid. Molar concentration of the boronic acid derivative is the same as that of the p-tolylboronic acid.

### Example 1.11

Example 1.11 is performed following the procedure of Example 1.3, except that potassium p-tolyltrifluoroborate is used instead of p-tolylboronic acid. Molar concentration of the boronic acid derivative is the same as that of the p-tolylboronic acid.

### Example 1.12

Example 1.12 is performed following the procedure of Example 1.4, except that potassium p-tolyltrifluoroborate is used instead of p-tolylboronic acid. Molar concentration of the boronic acid derivative is the same as that of the p-tolylboronic acid.

### Example 1.13

Example 1.13 is performed following the procedure of Example 1.5, except that potassium p-tolyltrifluoroborate is used instead of p-tolylboronic acid. Molar concentration of the boronic acid derivative is the same as that of the p-tolylboronic acid.

### Example 1.14

Example 1.14 is performed following the procedure of Example 1.6, except that potassium p-tolyltrifluoroborate is used instead of p-tolylboronic acid. Molar concentration of the boronic acid derivative is the same as that of the p-tolylboronic acid.

### Example 1.15

Example 1.15 is performed following the procedure of Example 1.7, except that potassium p-tolyltrifluoroborate is used instead of p-tolylboronic acid. Molar concentration of the boronic acid derivative is the same as that of the p-tolylboronic acid.

### Example 1.16

Example 1.16 is performed following the procedure of Example 1.8, except that potassium p-tolyltrifluoroborate is used instead of p-tolylboronic acid. Molar concentration of the boronic acid derivative is the same as that of the p-tolylboronic acid.

### Example 1.17

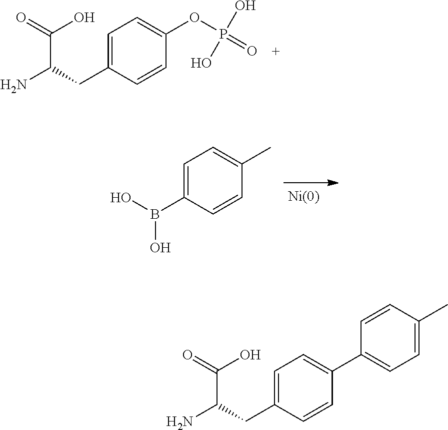

Tyrosine-O-phosphate (Tyr-O-Phos, 261 μg, 1 μmol) is suspended in dry THF, 10 μmol p-tolylboronic acid (1.36 mg), and potassium carbonate hydrate (K₂CO₃.H₂O, 691 μg, 5 μmol). To the reaction solution is added 0.1 μmol of a Ni (II) catalyst as a 10 μM stock solution in dry THF and 0.02 μmol tricyclohexyl phosphine, also as a THF stock solution. The reaction mixture is warmed to 50° C. with stirring. Reaction progression is monitored by liquid chromatography-mass spectrometry in which formation of the product is seen ([M+H]¹⁺=253.13 Da) with concurrent disappearance of the starting material, Tyr-O-Phos ([M+H]¹⁺=262.04 Da).

### Example 1.18

Example 1.18 is performed following the procedure of Example 1.17, except that potassium phosphate (K₃PO₄) hydrate is used as the base instead of potassium carbonate hydrate. Molar concentrations of the bases in both examples are kept the same.

### Example 1.19

Example 1.19 is performed following the procedure of Example 1.17, except that diisopropylethylamine (DIEA) is used as the base instead of potassium carbonate hydrate. Molar concentrations of the bases in both examples are kept the same.

### Example 1.20

Example 1.20 is performed following the procedure of Example 1.17, except that potassium hydroxide is used as the base instead of potassium carbonate hydrate. Molar concentrations of the bases in both examples are kept the same.

### Example 1.21

Example 1.21 is performed following the procedure of Example 1.17, except that potassium p-tolyltrifluoroborate is used instead of p-tolylboronic acid. Molar concentrations of the boron reactants in both examples are kept the same.

### Example 1.22

Example 1.22 is performed following the procedure of Example 1.18, except that potassium p-tolyltrifluoroborate is used instead of p-tolylboronic acid. Molar concentrations of the boron reactants in both examples are kept the same.

### Example 1.23

Example 1.23 is performed following the procedure of Example 1.19, except that potassium p-tolyltrifluoroborate is used instead of p-tolylboronic acid. Molar concentrations of the boron reactants in both examples are kept the same.

### Example 1.24

Example 1.24 is performed following the procedure of Example 1.20, except that potassium p-tolyltrifluoroborate is used instead of p-tolylboronic acid. Molar concentrations of the boron reactants in both examples are kept the same.

### Example 2: Preparation of Activated O-Phosphotyrosine for Use in Model Reactions

### Example 2.1: Preparation of the Activating Agent: Generation of Diazo-2,2,2-Trifluoroethane

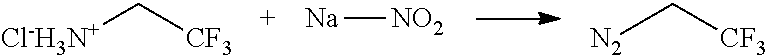

Generation of diazo-2,2,2-trifluoroethane is performed in an efficient fume hood. To a 2.0-ml plastic sample tube is added 100 μL N,N-DMF. Into this tube is placed an autosampler vial insert (200-300 μL capacity) containing 50 μL of a 1.0 mg/ml solution of 2,2,2-trifluoroethylamine hydrochloride in water (7.4 mM, 0.37 μmol). The sample tube is cooled on ice and to it added 50 μL of a 1.0 mg/ml solution of sodium nitrite in water (0.72 μmol) and the reaction chamber immediately closed and kept on ice for 30 minutes. The reaction tube is then brought to room temperature for 15 minutes before storing on ice for derivatization reactions. Diazo-2,2,2-trifluoroethane is generated as a gas within the autosampler vial insert and condenses in the N,N-DMF kept in the sample tube forming a brightly-colored solution. The inner autosampler vial insert is removed from the sample tube and discarded. The remaining reagent solution is used for 6 hours when kept on ice. Afterwards, remaining material is quenched in the hood with acetic acid until the solution turns colorless.

### Example 2.2: Derivatization of the Model Compound: Activation of O-Phosphotyrosine as its Tris-2,2,2-Trifluoroethyl Ester Analog

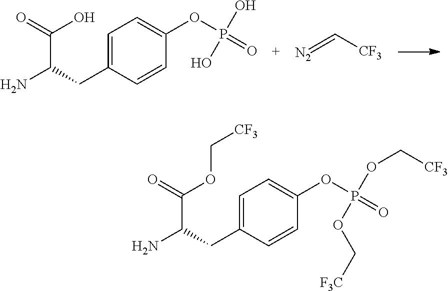

In a plastic sample tube in a fume hood, tyrosine-O-phosphate (261 μg, 1 μmol) is dissolved in 50 μL N,N-DMF and cooled on ice. To the solution is added 10 μL diazo-2,2,2-trifluoroethane solution in N,N-DMF at 0° C. prepared as described in the previous section. The reaction is allowed to stand on ice for 5 minutes, then brought to room temperature and allowed to stand for 30 minutes. Excess diazo-2,2,2-trifluoroethane is quenched by addition of dilute acetic acid solution in N,N-DMF until the reaction is colorless.

### Example 2.3: Model Systems Employing Activated 0-Phosphotyrosine and Arylboronic Acids

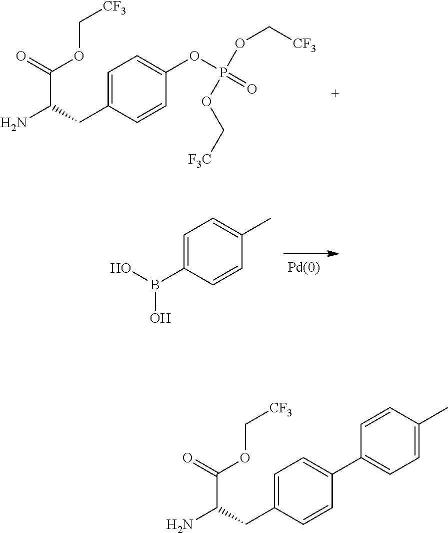

To 50 μL of tyrosine trifluoroethyl ester-O-[bis(2,2,2-trifluoroethoxy)] phosphate (1 μmol) solution in N,N-DMF is added 10 μmol p-tolylboronic acid (1.36 mg in 50 μL N,N-DMF), and potassium carbonate (K₂CO₃, 690 μg, 5 μmol in 10 μL H₂O). To the reaction solution is added 0.1 μmol of a PHOS palladium catalyst as a 10 μM stock solution in N,N-DMF. The reaction mixture is warmed to 50° C. with stirring. Reaction progression is monitored by liquid chromatography-mass spectrometry in which formation of the product is seen ([M+H]¹⁺=338.13 Da) with concurrent disappearance of the starting material, tyrosine trifluoroethyl ester-O-[bis(2,2,2-trifluoroethoxy)] phosphate ([M+H]¹⁺=508.05 Da).

### Example 2.4

Example 2.4 is performed following the procedure of Example 2.3, except that potassium phosphate is used instead of potassium carbonate. Molar concentration of the base is maintained under both conditions.

### Example 2.5

Example 2.5 is performed following the procedure of Example 2.3, except that diisopropylethylamine (DIEA) is used instead of potassium carbonate. Molar concentration of the base is maintained under both conditions.

### Example 2.6

Example 2.6 is performed following the procedure of Example 2.3, except that potassium hydroxide (KOH) is used instead of potassium carbonate. Molar concentration of the base is maintained under both conditions.

### Example 2.7

Example 2.7 is performed following the procedure of Example 2.3, except that the palladium catalyst is in the form of a pyridine-enhanced precatalyst preparation stabilization and initiation (PEPPSI) reagent. Molar concentration of the catalyst is maintained equal to that employed using the PHOS catalyst.

### Example 2.8

Example 2.8 is performed following the procedure of Example 2.4, except that the palladium catalyst is in the form of a pyridine-enhanced precatalyst preparation stabilization and initiation (PEPPSI) reagent. Molar concentration of the catalyst is maintained equal to that employed using the PHOS catalyst.

### Example 2.9

Example 2.9 is performed following the procedure of Example 2.5, except that the palladium catalyst is in the form of a pyridine-enhanced precatalyst preparation stabilization and initiation (PEPPSI) reagent. Molar concentration of the catalyst is maintained equal to that employed using the PHOS catalyst.

### Example 2.10

Example 2.10 is performed following the procedure of Example 2.6, except that the palladium catalyst is in the form of a pyridine-enhanced precatalyst preparation stabilization and initiation (PEPPSI) reagent. Molar concentration of the catalyst is maintained equal to that employed using the PHOS catalyst.

### Example 2.11

Example 2.11 is performed following the procedure of Example 2.3, except

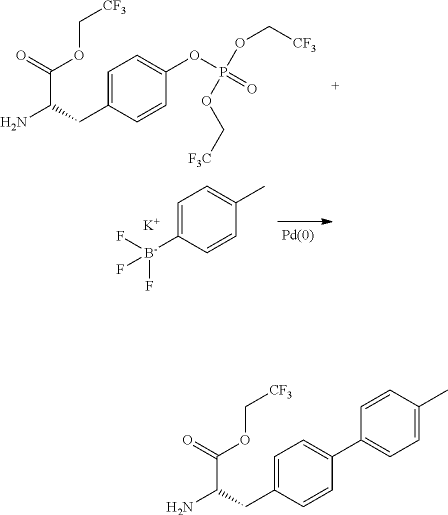

that potassium p-tolyltrifluoroborate is used instead of p-tolylboronic acid. Molar concentration of the boronic acid derivative is the same as that of the p-tolylboronic acid.

### Example 2.12

Example 2.12 is performed following the procedure of Example 2.4, except that potassium p-tolyltrifluoroborate is used instead of p-tolylboronic acid. Molar concentration of the boronic acid derivative is the same as that of the p-tolylboronic acid.

### Example 2.13

Example 2.13 is performed following the procedure of Example 2.5, except that potassium p-tolyltrifluoroborate is used instead of p-tolylboronic acid. Molar concentration of the boronic acid derivative is the same as that of the p-tolylboronic acid.

### Example 2.13

Example 2.13 is performed following the procedure of Example 2.6, except that potassium p-tolyltrifluoroborate is used instead of p-tolylboronic acid. Molar concentration of the boronic acid derivative is the same as that of the p-tolylboronic acid.

### Example 2.15

Example 2.15 is performed following the procedure of Example 2.7, except that potassium p-tolyltrifluoroborate is used instead of p-tolylboronic acid. Molar concentration of the boronic acid derivative is the same as that of the p-tolylboronic acid.

### Example 2.16

Example 2.16 is performed following the procedure of Example 2.8, except that potassium p-tolyltrifluoroborate is used instead of p-tolylboronic acid. Molar concentration of the boronic acid derivative is the same as that of the p-tolylboronic acid.

### Example 2.17

Example 2.17 is performed following the procedure of Example 2.9, except that potassium p-tolyltrifluoroborate is used instead of p-tolylboronic acid. Molar concentration of the boronic acid derivative is the same as that of the p-tolylboronic acid.

### Example 2.18

Example 2.18 is performed following the procedure of Example 2.10, except that potassium p-tolyltrifluoroborate is used instead of p-tolylboronic acid. Molar concentration of the boronic acid derivative is the same as that of the p-tolylboronic acid.

Example 2.19:

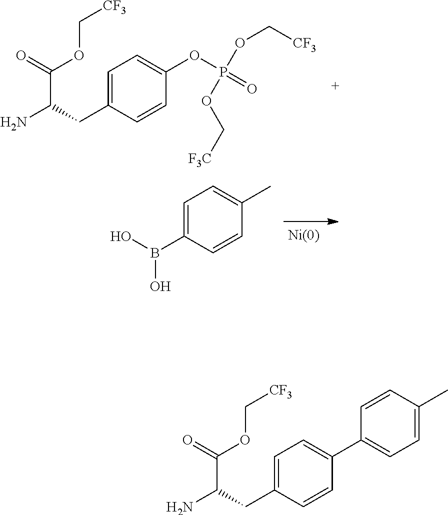

Tyrosine trifluoroethyl ester-O-[bis(2,2,2-trifluoroethoxy)] phosphate (1 μmol) solution in N,N-DMF is suspended in dry THF, 10 μmol p-tolylboronic acid (1.36 mg), and potassium carbonate hydrate (K₂CO₃—H₂O, 691 μg, 5 μmol). To the reaction solution is added 0.1 μmol of a Ni (II) catalyst as a 10 μM stock solution in dry THF and 0.02 μmol tricyclohexyl phosphine, also as a THF stock solution. The reaction mixture is warmed to 50° C. with stirring. Reaction progression is monitored by liquid chromatography-mass spectrometry in which formation of the product is seen ([M+H]¹⁺=338.13.13 Da) with concurrent disappearance of the starting material, tyrosine trifluoroethyl ester-O-[bis(2,2,2-trifluoroethoxy)] phosphate ([M+H]¹⁺=508.05 Da).

### Example 2.20

Example 2.20 is performed following the procedure of Example 2.19, except that potassium phosphate is used instead of potassium carbonate. Molar concentration of the base is maintained.

### Example 2.21

Example 2.21 is performed following the procedure of Example 2.19, except that diisopropylethylamine (DIEA) is used instead of potassium carbonate. Molar concentration of the base is maintained.

### Example 2.22

Example 2.22 is performed following the procedure of Example 2.19, except that potassium hydroxide (KOH) is used instead of potassium carbonate. Molar concentration of the base is maintained.

### Example 2.23

Example 2.23 is performed following the procedure of Example 2.19, except that potassium p-tolyltrifluoroborate is used instead of p-tolylboronic acid. Molar concentration of the boronic acid derivative is the same as that of the p-tolylboronic acid.

### Example 2.24

Example 2.24 is performed following the procedure of Example 2.20, except that potassium p-tolyltrifluoroborate is used instead of p-tolylboronic acid. Molar concentration of the boronic acid derivative is the same as that of the p-tolylboronic acid.

### Example 2.25

Example 2.25 is performed following the procedure of Example 2.21, except that potassium p-tolyltrifluoroborate is used instead of p-tolylboronic acid. Molar concentration of the boronic acid derivative is the same as that of the p-tolylboronic acid.

### Example 2.26

Example 2.26 is performed following the procedure of Example 2.22, except that potassium p-tolyltrifluoroborate is used instead of p-tolylboronic acid. Molar concentration of the boronic acid derivative is the same as that of the p-tolylboronic acid.

### Example 3: Model Systems Employing Small Molecules: Sonagashira Cross-Coupling Reactions

### Example 3.1

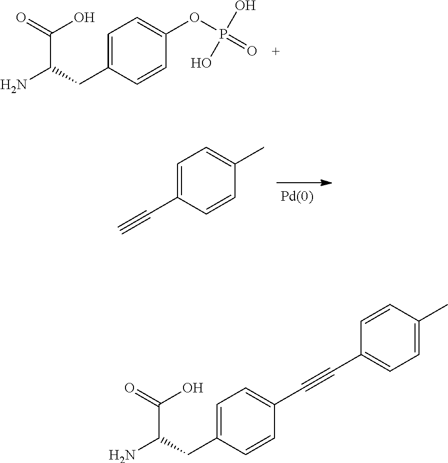

Tyrosine-O-phosphate (Tyr-O-Phos, 261 μg, 1 μmol) is dissolved in N,N-

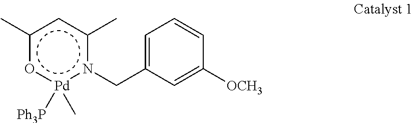

DMF, 10 μmol 4-ethynyltoluene (1.16 mg), and potassium carbonate (K₂CO₃, 690 μg, 5 μmol). To the reaction solution is added 0.1 μmol of a palladium (II) β-oxoiminatophosphane complex catalyst (catalyst 1, shown here) as a 10 μM stock solution in N,N-DMF. The reaction mixture is warmed to 50° C. with stirring. Reaction progression is monitored by liquid chromatography-mass spectrometry in which formation of the product is seen ([M+H]¹⁺=280.13 Da) with concurrent disappearance of the starting material, Tyr-O-Phos ([M+H]¹⁺=262.04 Da).

### Example 3.2

Example 3.2 is performed following the procedure of Example 3.1, except that triethylamine is used instead of potassium carbonate. Molar concentration of the base is maintained.

### Example 3.3

Example 3.3 is performed following the procedure of Example 3.1, except that a cesium carbonate is used instead of potassium carbonate. Molar concentration of the base is maintained.

### Example 3.4

Example 3.4 is performed following the procedure of Example 3.1, except that piperidine is used instead of potassium carbonate. Molar concentration of the base is maintained.

### Example 3.5

Example 3.5 is performed following the procedure of Example 3.1, except that acetonitrile is used as solvent instead of N,N-DMF, and PdCl₂(CH₃CN)₂/Xphos is used as the catalyst instead of palladium (II) β-oxoiminatophosphane complex. Molar concentration of the catalyst is maintained.

### Example 3.6

Example 3.6 is performed following the procedure of Example 3.2, except that acetonitrile is used as solvent instead of N,N-DMF, and PdCl₂(CH₃CN)₂/Xphos is used as the catalyst instead of palladium (II) β-oxoiminatophosphane complex. Molar concentration of the catalyst is maintained.

### Example 3.7

Example 3.7 is performed following the procedure of Example 3.3, except that that acetonitrile is used as solvent instead of N,N-DMF, and PdCl₂(CH₃CN)₂/Xphos is used as the catalyst instead of palladium (II) β-oxoiminatophosphane complex. Molar concentration of the catalyst is maintained.

### Example 3.8

Example 3.8 is performed following the procedure of Example 3.4, except that that acetonitrile is used as solvent instead of N,N-DMF, and PdCl₂(CH₃CN)₂/Xphos is used as the catalyst instead of palladium (II) β-oxoiminatophosphane complex. Molar concentration of the catalyst is maintained.

### Example 3.9

Example 3.9 is performed following the procedure of Example 3.1, except that PdCl₂(PPh₃)₂/AuCl(PPh₃) is used as the catalyst instead of palladium (II) 0-oxoiminatophosphane complex. Molar concentration of the catalyst is maintained.

### Example 3.10

Example 3.10 is performed following the procedure of Example 3.2, except that PdCl₂(PPh₃)₂/AuCl(PPh₃) is used as the catalyst instead of palladium (II) 0-oxoiminatophosphane complex. Molar concentration of the catalyst is maintained.

### Example 3.11

Example 3.11 is performed following the procedure of Example 3.3, except that PdCl₂(PPh₃)₂/AuCl(PPh₃) is used as the catalyst instead of palladium (II) 0-oxoiminatophosphane complex. Molar concentration of the catalyst is maintained.

### Example 3.12

Example 3.12 is performed following the procedure of Example 3.4, except that PdCl₂(PPh₃)₂/AuCl(PPh₃) is used as the catalyst instead of palladium (II) 0-oxoiminatophosphane complex. Molar concentration of the catalyst is maintained.

### Example 3.13

Example 3.13 is performed following the procedure of Example 3.1, except that DMSO is used as solvent instead of N,N-DMF, and PdCl₂(PCy₃)₂ is used as the catalyst instead of palladium (II) β-oxoiminatophosphane complex. Molar concentration of the catalyst is maintained.

### Example 3.14

Example 3.14 is performed following the procedure of Example 3.2, except that PdCl₂(PCy₃)₂ is used as the catalyst instead of palladium (II) β-oxoiminatophosphane complex. Molar concentration of the catalyst is maintained.

### Example 3.15

Example 3.15 is performed following the procedure of Example 3.3, except that DMSO is used as solvent instead of N,N-DMF, and PdCl₂(PCy₃)₂ is used as the catalyst instead of palladium (II) β-oxoiminatophosphane complex. Molar concentration of the catalyst is maintained.

### Example 3.16

Example 3.16 is performed following the procedure of Example 3.4, except that DMSO is used as solvent instead of N,N-DMF, and PdCl₂(PCy₃)₂ is used as the catalyst instead of palladium (II) β-oxoiminatophosphane complex. Molar concentration of the catalyst is maintained.

### Example 3.17

Example 3.17 is performed following the procedure of Example 3.1, except that it is performed in acetonitrile instead of N,N-DMF, and that a mixture of palladacycle 1

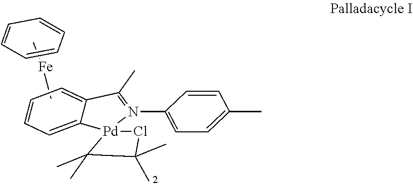

and Xphos is used as the catalyst instead of palladium (II) (3-oxoiminatophosphane complex. Molar concentration of the catalyst is maintained. The structure of palladacycle 1 is shown.

### Example 3.18

Example 3.18 is performed following the procedure of Example 3.2, except that it is performed in acetonitrile instead of N,N-DMF, and that a mixture of palladacycle 1 and Xphos is used as the catalyst instead of palladium (II) β-oxoiminatophosphane complex. Molar concentration of the catalyst is maintained.

### Example 3.19

Example 3.19 is performed following the procedure of Example 3.3, except that it is performed in acetonitrile instead of N,N-DMF, and that a mixture of palladacycle 1 and Xphos is used as the catalyst instead of palladium (II) β-oxoiminatophosphane complex. Molar concentration of the catalyst is maintained.

### Example 3.20

Example 3.20 is performed following the procedure of Example 3.4, except that it is performed in acetonitrile instead of N,N-DMF, and that a mixture of palladacycle 1 and Xphos is used as the catalyst instead of palladium (II) β-oxoiminatophosphane complex. Molar concentration of the catalyst is maintained.

### Example 3.21

Example 3.21 is performed following the procedure of Example 3.1, except that Tyr-O-phosphate-tris-trifluoroethyl ester is used instead of Tyr-O-phosphate as a reactant. Otherwise, all relative molar concentrations and conditions are maintained. Tyr-O-phosphate-tris-trifluoroethyl ester is prepared as described above.

### Example 3.22

Example 3.22 is performed following the procedure of Example 3.2, except that Tyr-O-phosphate-tris-trifluoroethyl ester is used instead of Tyr-O-phosphate as a reactant. Otherwise, all relative molar concentrations and conditions are maintained. Tyr-O-phosphate-tris-trifluoroethyl ester is prepared as described above.

### Example 3.23

Example 3.23 is performed following the procedure of Example 3.3, except that Tyr-O-phosphate-tris-trifluoroethyl ester is used instead of Tyr-O-phosphate as a reactant. Otherwise, all relative molar concentrations and conditions are maintained. Tyr-O-phosphate-tris-trifluoroethyl ester is prepared as described above.

### Example 3.24

Example 3.24 is performed following the procedure of Example 3.4, except that Tyr-O-phosphate-tris-trifluoroethyl ester is used instead of Tyr-O-phosphate as a reactant. Otherwise, all relative molar concentrations and conditions are maintained. Tyr-O-phosphate-tris-trifluoroethyl ester is prepared as described above.

### Example 3.25

Example 3.25 is performed following the procedure of Example 3.5, except that Tyr-O-phosphate-tris-trifluoroethyl ester is used instead of Tyr-O-phosphate as a reactant. Otherwise, all relative molar concentrations and conditions are maintained. Tyr-O-phosphate-tris-trifluoroethyl ester is prepared as described above.

### Example 3.26

Example 3.26 is performed following the procedure of Example 3.6, except that Tyr-O-phosphate-tris-trifluoroethyl ester is used instead of Tyr-O-phosphate as a reactant. Otherwise, all relative molar concentrations and conditions are maintained. Tyr-O-phosphate-tris-trifluoroethyl ester is prepared as described above.

### Example 3.27

Example 3.27 is performed following the procedure of Example 3.7, except that Tyr-O-phosphate-tris-trifluoroethyl ester is used instead of Tyr-O-phosphate as a reactant. Otherwise, all relative molar concentrations and conditions are maintained. Tyr-O-phosphate-tris-trifluoroethyl ester is prepared as described above.

### Example 3.28

Example 3.28 is performed following the procedure of Example 3.8, except that Tyr-O-phosphate-tris-trifluoroethyl ester is used instead of Tyr-O-phosphate as a reactant. Otherwise, all relative molar concentrations and conditions are maintained. Tyr-O-phosphate-tris-trifluoroethyl ester is prepared as described above.

### Example 3.29

Example 3.29 is performed following the procedure of Example 3.9, except that Tyr-O-phosphate-tris-trifluoroethyl ester is used instead of Tyr-O-phosphate as a reactant. Otherwise, all relative molar concentrations and conditions are maintained. Tyr-O-phosphate-tris-trifluoroethyl ester is prepared as described above.

### Example 3.30

Example 3.30 is performed following the procedure of Example 3.10, except that Tyr-O-phosphate-tris-trifluoroethyl ester is used instead of Tyr-O-phosphate as a reactant. Otherwise, all relative molar concentrations and conditions are maintained. Tyr-O-phosphate-tris-trifluoroethyl ester is prepared as described above.

### Example 3.31

Example 3.31 is performed following the procedure of Example 3.11, except that Tyr-O-phosphate-tris-trifluoroethyl ester is used instead of Tyr-O-phosphate as a reactant. Otherwise, all relative molar concentrations and conditions are maintained. Tyr-O-phosphate-tris-trifluoroethyl ester is prepared as described above.

### Example 3.32

Example 3.32 is performed following the procedure of Example 3.12, except that Tyr-O-phosphate-tris-trifluoroethyl ester is used instead of Tyr-O-phosphate as a reactant. Otherwise, all relative molar concentrations and conditions are maintained. Tyr-O-phosphate-tris-trifluoroethyl ester is prepared as described above.

### Example 3.33

Example 3.33 is performed following the procedure of Example 3.13, except that Tyr-O-phosphate-tris-trifluoroethyl ester is used instead of Tyr-O-phosphate as a reactant. Otherwise, all relative molar concentrations and conditions are maintained. Tyr-O-phosphate-tris-trifluoroethyl ester is prepared as described above.

### Example 3.34

Example 3.34 is performed following the procedure of Example 3.14, except that Tyr-O-phosphate-tris-trifluoroethyl ester is used instead of Tyr-O-phosphate as a reactant. Otherwise, all relative molar concentrations and conditions are maintained. Tyr-O-phosphate-tris-trifluoroethyl ester is prepared as described above.

### Example 3.35

Example 3.35 is performed following the procedure of Example 3.15, except that Tyr-O-phosphate-tris-trifluoroethyl ester is used instead of Tyr-O-phosphate as a reactant. Otherwise, all relative molar concentrations and conditions are maintained. Tyr-O-phosphate-tris-trifluoroethyl ester is prepared as described above.

### Example 3.36

Example 3.36 is performed following the procedure of Example 3.16, except that Tyr-O-phosphate-tris-trifluoroethyl ester is used instead of Tyr-O-phosphate as a reactant. Otherwise, all relative molar concentrations and conditions are maintained. Tyr-O-phosphate-tris-trifluoroethyl ester is prepared as described above.

### Example 3.37

Example 3.37 is performed following the procedure of Example 3.17, except that Tyr-O-phosphate-tris-trifluoroethyl ester is used instead of Tyr-O-phosphate as a reactant. Otherwise, all relative molar concentrations and conditions are maintained. Tyr-O-phosphate-tris-trifluoroethyl ester is prepared as described above.

### Example 3.38

Example 3.38 is performed following the procedure of Example 3.18, except that Tyr-O-phosphate-tris-trifluoroethyl ester is used instead of Tyr-O-phosphate as a reactant. Otherwise, all relative molar concentrations and conditions are maintained. Tyr-O-phosphate-tris-trifluoroethyl ester is prepared as described above.

### Example 3.39

Example 3.39 is performed following the procedure of Example 3.19, except that Tyr-O-phosphate-tris-trifluoroethyl ester is used instead of Tyr-O-phosphate as a reactant. Otherwise, all relative molar concentrations and conditions are maintained. Tyr-O-phosphate-tris-trifluoroethyl ester is prepared as described above.

### Example 3.40

Example 3.40 is performed following the procedure of Example 3.20, except that Tyr-O-phosphate-tris-trifluoroethyl ester is used instead of Tyr-O-phosphate as a reactant. Otherwise, all relative molar concentrations and conditions are maintained. Tyr-O-phosphate-tris-trifluoroethyl ester is prepared as described above.

### Example 4: Adaptation of Reaction Conditions for Suzuki Cross-Coupling Reactions of Arylboronic Acids and O-Phosphotyrosine-Containing Peptides

### Example 4.1: Preparation of O-Phosphotyrosine-Conjugated Peptides

Phosphotyrosine-conjugated bovine serum albumin (BSA-pTyr, 500 μmol, approximately 35 μg protein) is dissolved in 250 μL of 50 mM ammonium bicarbonate (pH=8.0-8.5) containing 4 M guanidinium chloride. To this solution is added 0.7 μg proteomics-grade trypsin and the reaction solution allowed to sit overnight at room temperature. Afterwards, the solution is acidified to a final concentration of 0.5% trifluoroacetic acid and the resulting peptides freed from buffer and chaotropes using a C-18 solid-phase extraction cartridge. Solvent is removed using reduced pressure. Peptides are identified by LC-MS/MS using peptide identification software such as Andromeda or Mascot.

### Example 4.2: Adaptation of Suzuki Cross-Coupling Reactions Between an Arylboronic Acid and Peptides Containing Unactivated O-Phosphotyrosine

Purified peptides containing O-phosphotyrosine residues are subjected to conditions adapted for reactions on small molecules as determined from Examples 1-24. The resulting mixture of peptides is freed from reagent additives using a C-18 solid-phase extraction cartridge before drying under reduced pressure. The resulting dried material is analyzed by LC-MS/MS using peptide identification software such as Andromeda or Mascot.

### Example 4.3: Adaptation of Suzuki Cross-Coupling Reactions Between an Arylboronic Acid and Peptides Containing Activated O-Phosphotyrosine

Purified peptides containing O-phosphotyrosine residues are derivatized as their 2,2,2-trifluoroethyl diester analogs using the conditions described above for O-phosphotyrosine. These peptides are subjected to conditions adapted for Suzuki cross-coupling reactions as determined in Examples 25-48. The resulting mixture of peptides is freed from reagent additives using a C-18 solid-phase extraction cartridge before drying under reduced pressure. The resulting dried material is analyzed by LC-MS/MS using peptide identification software such as Andromeda or Mascot.

### Example 5: Adaptation of Reaction Conditions for Sonogashira Cross-Coupling Reactions of Alkynes and O-Phosphotyrosine-Containing Peptides

### Example 5.1: Adaptation of Sonagashira Cross-Coupling Reactions Between an Alkyne and Peptides Containing Unactivated O-Phosphotyrosine

Purified peptides containing O-phosphotyrosine residues are subjected to conditions adapted for reactions on small molecules as determined from Examples 49-68. The resulting mixture of peptides is freed from reagent additives using a C-18 solid-phase extraction cartridge before drying under reduced pressure. The resulting dried material is analyzed by LC-MS/MS using peptide identification software such as Andromeda or Mascot.

### Example 5.2: Adaptation of Sonagashira Cross-Coupling Reactions Between an Alkyne and Peptides Containing Activated O-Phosphotyrosine

Purified peptides containing O-phosphotyrosine residues are derivatized as their 2,2,2-trifluoroethyl diester analogs using the conditions described above for O-phosphotyrosine. These peptides are subjected to conditions adapted for Suzuki cross-coupling reactions as determined in Examples 69-88. The resulting mixture of peptides is freed from reagent additives using a C-18 solid-phase extraction cartridge before drying under reduced pressure. The resulting dried material is analyzed by LC-MS/MS using peptide identification software such as Andromeda or Mascot.

### Example 6: Adaptation of Reaction Conditions for Suzuki Cross-Coupling Reactions of Arylboronic Acids and O-Phosphotyrosine-Containing Proteins

### Example 6.1: Adaptation of Suzuki Cross-Coupling Reactions Between an Arylboronic Acid and Protein Containing Unactivated O-Phosphotyrosine

Phosphotyrosine-conjugated bovine serum albumin (BSA-pTyr, 500 μmol, approximately 35 μg protein) is dissolved in 250 μL of N,N-DMF and subjected to conditions adapted for reactions on peptides as determined from Example 89. The resulting protein sample is purified by sodium dodecyl sulfate polyacrylamide gel electrophoresis (SDS-PAGE) and subjected to in-gel digestion using trypsin. Peptides extracted from the gel pieces are purified using a C-18 solid-phase extraction cartridge and analyzed by LC-MS/MS using peptide identification software such as Andromeda or Mascot.

### Example 6.2: Adaptation of Suzuki Cross-Coupling Reactions Between an Arylboronic Acid and Protein Containing Activated O-Phosphotyrosine

Phosphotyrosine-conjugated bovine serum albumin (BSA-pTyr, 500 μmol, approximately 35 μg protein) is dissolved in 250 μL of N,N-DMF and derivatized as its 0-phosphotyrosine 2,2,2-trifluoroethyl diester analogs using the conditions described above for O-phosphotyrosine. It is then subjected to conditions adapted for reactions on peptides as determined from Example 90. The resulting protein sample is purified by SDS-PAGE and subjected to in-gel digestion using trypsin. Peptides extracted from the gel pieces are purified using a C-18 solid-phase extraction cartridge and analyzed by LC-MS/MS using peptide identification software such as Andromeda or Mascot.

### Example 7: Adaptation of Reaction Conditions for Sonogashira Cross-Coupling Reactions of Alkynes and O-Phosphotyrosine-Containing Peptides

### Example 7.1: Adaptation of Sonogashira Cross-Coupling Reactions Between an Alkyne and Protein Containing Unactivated O-Phosphotyrosine

Phosphotyrosine-conjugated bovine serum albumin (BSA-pTyr, 500 μmol, approximately 35 μg protein) is dissolved in 250 μL of N,N-DMF and subjected to conditions adapted for reactions on peptides as determined from Example 91. The resulting protein sample is purified by sodium dodecyl sulfate polyacrylamide gel electrophoresis (SDS-PAGE) and subjected to in-gel digestion using trypsin. Peptides extracted from the gel pieces are purified using a C-18 solid-phase extraction cartridge and analyzed by LC-MS/MS using peptide identification software such as Andromeda or Mascot.

### Example 7.2: Adaptation of Sonogashira Cross-Coupling Reactions Between an Alkyne and Protein Containing Activated O-Phosphotyrosine

Phosphotyrosine-conjugated bovine serum albumin (BSA-pTyr, 500 μmol, approximately 35 μg protein) is dissolved in 250 μL of N,N-DMF and derivatized as its O-phosphotyrosine 2,2,2-trifluoroethyl diester analogs using the conditions described above for O-phosphotyrosine. It is then subjected to conditions adapted for reactions on peptides as determined from Example 92. The resulting protein sample is purified by SDS-PAGE and subjected to in-gel digestion using trypsin. Peptides extracted from the gel pieces are purified using a C-18 solid-phase extraction cartridge and analyzed by LC-MS/MS using peptide identification software such as Andromeda or Mascot.

### Example 8

Reagents, Solvents, Catalysts, Bases, and Reaction Temperatures of Suzuki Reactions and Sonogashira Reactions according to various examples herein are shown in Table I below.

### Tyrosine Phosphorylation Analysis of Biological Samples

Tyrosine Phosphorylation Analysis Performed on Whole-Protein Biological Samples: Protein Samples Derived from Cell Culture

### Example 97

A cultured cell sample is harvested by conventional methods. For example, when live cell samples are grown on a sterile surface, growth media is removed and cells harvested by scraping in the presence of ice-cold lysis buffer (for example, B-PER Complete buffer) containing a protease inhibitor cocktail and a phosphatase inhibitor cocktail. When cell samples are grown in a suspension culture, the sample is supplemented with a protease inhibitor cocktail and a phosphatase inhibitor cocktail and placed on ice. Cells are then pelleted by centrifugation. They are homogenized in the presence of ice-cold lysis buffer containing a protease inhibitor cocktail and a phosphatase inhibitor cocktail. Samples are homogenized at 0-4° C. by conventional means, including the use of a Dounce homogenizer, sonication, French press, “bead-beater,” or commercially-available cell disruptors. Protein samples so obtained are precipitated by any one of several methods including the use of acids (e.g. trichloroacetic acid), organic cosolvents, detergent entanglement, or chloroform/methanol.

Proteins are redissolved in a solvent system containing cross-coupling reactants and reagents optimized for reaction conditions according to the protocols developed using model systems. In this case, the coupling moiety is part of a reporter label such as a biotinylated linker, fluorescent dye, or isotope label. Proteins are then fractionated by any one of several methods such as SDS-PAGE, chromatography, or capillary electrophoresis. Labeled proteins are then enriched by affinity chromatography (for example, biotinylated proteins captured using immobilized avidin), or observed using a corresponding detector, such as a gel analyzer equipped with a fluorescence detector (in the case of fluorescently-labeled proteins). Alternatively, biotinylated proteins can be analyzed by Western blot analysis of the labeled protein sample using an avidin-peroxidase conjugate for detection.

Tyrosine Phosphorylation Analysis Performed on Whole-Protein Biological Samples: Protein Samples Derived from Biological Tissue

### Example 98

Procedures for example 98 are performed following the procedures used in example 97. However, the alternative of flash-freezing tissue samples in liquid nitrogen and grinding them as a frozen powder using a liquid nitrogen-cooled mortar and pestle for sample disruption is also possible in this case, a method rarely used for cultured cell samples.

Tyrosine Phosphorylation Analysis Performed on Peptides Derived from Biological Samples: Peptide Samples Derived from Cell Culture

### Example 99

Protein samples are prepared according to the procedures used in example 97. After precipitation, proteins are proteolyzed to their constituent peptides using a proteomics-grade protease such as trypsin, Lys-C, or Asp-N. Resultant peptides are purified using solid-phase extraction on a C-18 cartridge and solvent removed under reduced pressure.

Peptides are redissolved in a solvent system containing cross-coupling reactants and reagents optimized for reaction conditions according to the protocols developed using model systems. In this case, the coupling moiety is part of a reporter label such as a biotinylated linker, fluorescent dye, or isotope label. Labeled peptides are then enriched by affinity chromatography (for example, biotinylated proteins captured using immobilized avidin), or observed using by a separation technique coupled to an appropriate detector, such as such as HPLC or capillary electrophoresis coupled to a fluorescence detector. In all cases, labeled peptides are identified by LC-MS/MS using peptide analysis software such as Andromeda or Mascot.

Tyrosine Phosphorylation Analysis Performed on Peptides Derived from Biological Samples: Peptide Samples Derived from Cell Culture Example 100:

Protein preparation procedures for example 100 are performed following the procedures used in example 98. However, the alternative of flash-freezing tissue samples in liquid nitrogen and grinding them as a frozen powder using a liquid nitrogen-cooled mortar and pestle for sample disruption is also possible in this case, a method rarely used for cultured cell samples.

After precipitation, proteins are proteolyzed to their constituent peptides using a proteomics-grade protease such as trypsin, Lys-C, or Asp-N. Resultant peptides are purified using solid-phase extraction on a C-18 cartridge and solvent removed under reduced pressure.

Peptides are redissolved in a solvent system containing cross-coupling reactants and reagents optimized for reaction conditions according to the protocols developed using model systems. In this case, the coupling moiety is part of a reporter label such as a biotinylated linker, fluorescent dye, or isotope label. Labeled peptides are then enriched by affinity chromatography (for example, biotinylated proteins captured using immobilized avidin), or observed using by a separation technique coupled to an appropriate detector, such as such as HPLC or capillary electrophoresis coupled to a fluorescence detector. In all cases, labeled peptides are identified by LC-MS/MS using peptide analysis software such as Andromeda or Mascot.

TABLE I

| | | | | | | |
|---|---|---|---|---|---|---|
| Ex- | Aryl | | | | | Temp- |
| ample | phosphate | Reagent | Solvent | Catalyst | Base | erature |

Suzuki Reactions

| | | | | | | |
|---|---|---|---|---|---|---|
| 1.1 | Tyr-O- | p-tolylboronic | N,N- | Palladium PHOS | potassium | 50° C. |
| | phosphate | acid | DMF/H₂O | reagent | carbonate | |
| 1.2 | Tyr-O- | p-tolylboronic | N,N- | Palladium PHOS | potassium | 50° C. |
| | phosphate | acid | DMF/H₂O | regent | phosphate | |
| | | | | | (K₃PO₄) | |
| 1.3 | Tyr-O- | p-tolylboronic | N,N- | Palladium PHOS | diisopropyl | 50° C. |
| | phosphate | acid | DMF/H₂O | reagent | ethylamine | |
| | | | | | (DIEA) | |
| 1.4 | Tyr-O- | p-tolylboronic | N,N- | Palladium PHOS | potassium | 50° C. |
| | phosphate | acid | DMF/H₂O | reagent | hydroxide | |
| 1.5 | Tyr-O- | p-tolylboronic | N,N- | Palladium PEPPSI | potassium | 50° C. |
| | phosphate | acid | DMF/H₂O | reagent | carbonate | |
| 1.6 | Tyr-O- | p-tolylboronic | N,N- | Palladium PEPPSI | potassium | 50° C. |
| | phosphate | acid | DMF/H₂O | reagent | phosphate | |
| | | | | | (K₃PO₄) | |
| 1.7 | Tyr-O- | p-tolylboronic | N,N- | Palladium PEPPSI | diisopropyl | 50° C. |
| | phosphate | acid | DMF/H₂O | reagent | ethylamine | |
| | | | | | (DIEA) | |
| 1.8 | Tyr-O- | p-tolylboronic | N,N- | Palladium PEPPSI | potassium | 50° C. |
| | phosphate | acid | DMF/H₂O | reagent | hydroxide | |
| 1.9 | Tyr-O- | potassium p- | N,N- | Palladium PHOS | potassium | 50° C. |
| | phosphate | tolyltrifluoro- | DMF/H₂O | reagent | carbonate | |
| | | borate | | | | |
| 1.10 | Tyr-O- | potassium p- | N,N- | Palladium PHOS | potassium | 50° C. |
| | phosphate | tolyltrifluoro- | DMF/H₂O | reagent | phosphate | |
| | | borate | | | (K₃PO₄) | |
| 1.11 | Tyr-O- | potassium p- | N,N- | Palladium PHOS | diisopropyl | 50° C. |
| | phosphate | tolyltrifluoro- | DMF/H₂O | reagent | ethylamine | |
| | | borate | | | (DIEA) | |
| 1.12 | Tyr-O- | potassium p- | N,N- | Palladium PHOS | potassium | 50° C. |
| | phosphate | tolyltrifluoro- | DMF/H₂O | reagent | hydroxide | |
| | | borate | | | | |
| 1.13 | Tyr-O- | potassium p- | N,N- | Palladium PEPPSI | potassium | 50° C. |
| | phosphate | tolyltrifluoro- | DMF/H₂O | reagent | carbonate | |
| | | borate | | | | |
| 1.14 | Tyr-O- | potassium p- | N,N- | Palladium PEPPSI | potassium | 50° C. |
| | phosphate | tolyltrifluoro- | DMF/H₂O | reagent | phosphate | |
| | | borate | | | (K₃PO₄) | |
| 1.15 | Tyr-O- | potassium p- | N,N- | Palladium PEPPSI | diisopropyl | 50° C. |
| | phosphate | tolyltrifluoro- | DMF/H₂O | reagent | ethylamine | |
| | | borate | | | (DIEA) | |
| 1.16 | Tyr-O- | potassium p- | N,N- | Palladium PEPPSI | potassium | 50° C. |
| | phosphate | tolyltrifluoro- | DMF/H₂O | reagent | hydroxide | |
| | | borate | | | | |
| 1.17 | Tyr-O- | p-tolylboronic | THF | Ni^{II}Cl(1- | potassium | 50° C. |
| | phosphate | acid | | naphthyl)(PCy₃)₂/ | carbonate | |
| | | | | PCy₃ σ-complex | hydrate | |
| 1.18 | Tyr-O- | p-tolylboronic | THF | Ni^{II}Cl(1- | potassium | 50° C. |
| | phosphate | acid | | naphthyl)(PCy₃)₂/ | phosphate | |
| | | | | PCy₃ σ-complex | (K₃PO₄) | |
| | | | | | hydrate | |
| 1.19 | Tyr-O- | p-tolylboronic | THF | Ni^{II}Cl(1- | diisopropyl | 50° C. |
| | phosphate | acid | | naphthyl)(PCy₃)₂/ | ethylamine | |
| | | | | PCy₃ σ-complex | (DIEA) | |
| 1.20 | Tyr-O- | p-tolylboronic | THF | Ni^{II}Cl(1- | potassium | 50° C. |
| | phosphate | acid | | naphthyl)(PCy₃)₂/ | hydroxide | |
| | | | | PCy₃ σ-complex | | |
| 1.21 | Tyr-O- | potassium p- | THF | Ni^{II}Cl(1- | potassium | 50° C. |
| | phosphate | tolyltrifluoro- | | naphthyl)(PCy₃)₂/ | carbonate | |
| | | borate | | PCy₃ σ-complex | hydrate | |
| 1.22 | Tyr-O- | potassium p- | THF | Ni^{II}Cl(1- | potassium | 50° C. |
| | phosphate | tolyltrifluoro- | | naphthyl)(PCy₃)₂/ | phosphate | |
| | | borate | | PCy₃ σ-complex | (K₃PO₄) | |
| | | | | | hydrate | |
| 1.23 | Tyr-O- | potassium p- | THF | Ni^{II}Cl(1- | diisopropyl | 50° C. |
| | phosphate | tolyltrifluoro- | | naphthyl)(PCy₃)₂/ | ethylamine | |
| | | borate | | PCy₃ σ-complex | (DIEA) | |
| 1.24 | Tyr-O- | potassium p- | THF | Ni^{II}Cl(1- | potassium | 50° C. |
| | phosphate | tolyltrifluoro- | | naphthyl)(PCy₃)₂/ | hydroxide | |
| | | borate | | PCy₃ σ-complex | | |
| 2.3 | Tyr-O- | p-tolylboronic | N,N- | Palladium PHOS | potassium | 50° C. |
| | phosphate- | acid | DMF/H₂O | reagent | carbonate | |
| | tris- | | | | | |
| | trifluoroethyl | | | | | |
| | ester | | | | | |
| 2.4 | Tyr-O- | p-tolylboronic | N,N- | Palladium PHOS | potassium | 50° C. |
| | phosphate- | acid | DMF/H₂O | reagent | phosphate | |
| | tris- | | | | (K₃PO₄) | |
| | trifluoroethyl | | | | | |
| | ester | | | | | |
| 2.5 | Tyr-O- | p-tolylboronic | N,N- | Palladium PHOS | diisopropyl | 50° C. |
| | phosphate- | acid | DMF/H₂O | reagent | ethylamine | |
| | tris- | | | | (DIEA) | |
| | trifluoroethyl | | | | | |
| | ester | | | | | |
| 2.6 | Tyr-O- | p-tolylboronic | N,N- | Palladium PHOS | potassium | 50° C. |
| | phosphate- | acid | DMF/H₂O | reagent | hydroxide | |
| | tris- | | | | | |
| | trifluoroethyl | | | | | |
| | ester | | | | | |
| 2.7 | Tyr-O- | p-tolylboronic | N,N- | Palladium PEPPSI | potassium | 50° C. |
| | phosphate- | acid | DMF/H₂O | reagent | carbonate | |
| | tris- | | | | | |
| | trifluoroethyl | | | | | |
| | ester | | | | | |
| 2.8 | Tyr-O- | p-tolylboronic | N,N- | Palladium PEPPSI | potassium | 50° C. |
| | phosphate- | acid | DMF/H₂O | reagent | phosphate | |
| | tris- | | | | (K₃PO₄) | |
| | trifluoroethyl | | | | | |
| | ester | | | | | |
| 2.9 | Tyr-O- | p-tolylboronic | N,N- | Palladium PEPPSI | diisopropyl | 50° C. |
| | phosphate- | acid | DMF/H₂O | reagent | ethylamine | |
| | tris- | | | | (DIEA) | |
| | trifluoroethyl | | | | | |
| | ester | | | | | |
| 2.10 | Tyr-O- | p-tolylboronic | N,N- | Palladium PEPPSI | potassium | 50° C. |
| | phosphate- | acid | DMF/H₂O | reagent | hydroxide | |
| | tris- | | | | | |
| | trifluoroethyl | | | | | |
| | ester | | | | | |
| 2.11 | Tyr-O- | potassium p- | N,N- | Palladium PHOS | potassium | 50° C. |
| | phosphate- | tolyltrifluoro- | DMF/H₂O | reagent | carbonate | |
| | tris- | borate | | | | |
| | trifluoroethyl | | | | | |
| | ester | | | | | |
| 2.12 | Tyr-O- | potassium p- | N,N- | Palladium PHOS | potassium | 50° C. |
| | phosphate- | tolyltrifluoro- | DMF/H₂O | reagent | phosphate | |
| | tris- | borate | | | (K₃PO₄) | |
| | trifluoroethyl | | | | | |
| | ester | | | | | |
| 2.13 | Tyr-O- | potassium p- | N,N- | Palladium PHOS | diisopropyl | 50° C. |
| | phosphate- | tolyltrifluoro- | DMF/H₂O | reagent | ethylamine | |
| | tris- | borate | | | (DIEA) | |
| | trifluoroethyl | | | | | |
| | ester | | | | | |
| 2.14 | Tyr-O- | potassium p- | N,N- | Palladium PHOS | potassium | 50° C. |
| | phosphate- | tolyltrifluoro- | DMF/H₂O | reagent | hydroxide | |
| | tris- | borate | | | | |
| | trifluoroethyl | | | | | |
| | ester | | | | | |
| 2.15 | Tyr-O- | potassium p- | N,N- | Palladium PEPPSI | potassium | 50° C. |
| | phosphate- | tolyltrifluoro- | DMF/H₂O | reagent | carbonate | |
| | tris- | borate | | | | |
| | trifluoroethyl | | | | | |
| | ester | | | | | |
| 2.16 | Tyr-O- | potassium p- | N,N- | Palladium PEPPSI | potassium | 50° C. |
| | phosphate- | tolyltrifluoro- | DMF/H₂O | reagent | phosphate | |
| | tris- | borate | | | (K₃PO₄) | |
| | trifluoroethyl | | | | | |
| | ester | | | | | |
| 2.17 | Tyr-O- | potassium p- | N,N- | Palladium PEPPSI | diisopropyl | 50° C. |
| | phosphate- | tolyltrifluoro- | DMF/H₂O | reagent | ethylamine | |
| | tris- | borate | | | (DIEA) | |
| | trifluoroethyl | | | | | |
| | ester | | | | | |
| 2.18 | Tyr-O- | potassium p- | N,N- | Palladium PEPPSI | potassium | 50° C. |
| | phosphate- | tolyltrifluoro- | DMF/H₂O | reagent | hydroxide | |
| | tris- | borate | | | | |
| | trifluoroethyl | | | | | |
| | ester | | | | | |
| 2.19 | Tyr-O- | p-tolylboronic | THF | Ni^{II}Cl(1- | potassium | 50° C. |
| | phosphate- | acid | | naphthyl)(PCy₃)₂/ | carbonate | |
| | tris- | | | PCy₃ σ-complex | hydrate | |
| | trifluoroethyl | | | | | |
| | ester | | | | | |
| 2.20 | Tyr-O- | p-tolylboronic | THF | Ni^{II}Cl(1- | potassium | 50° C. |
| | phosphate- | acid | | naphthyl)(PCy₃)₂/ | phosphate | |
| | tris- | | | PCy₃ σ-complex | (K₃PO₄) | |
| | trifluoroethyl | | | | hydrate | |
| | ester | | | | | |
| 2.21 | Tyr-O- | p-tolylboronic | THF | Ni^{II}Cl(1- | diisopropyl | 50° C. |
| | phosphate- | acid | | naphthyl)(PCy₃)₂/ | ethylamine | |
| | tris- | | | PCy₃ σ-complex | (DIEA) | |
| | trifluoroethyl | | | | | |
| | ester | | | | | |
| 2.22 | Tyr-O- | p-tolylboronic | THF | Ni^{II}Cl(1- | potassium | 50° C. |
| | phosphate- | acid | | naphthyl)(PCy₃)₂/ | hydroxide | |
| | tris- | | | PCy₃ σ-complex | | |
| | trifluoroethyl | | | | | |
| | ester | | | | | |
| 2.23 | Tyr-O- | potassium p- | THF | Ni^{II}Cl(1- | potassium | 50° C. |
| | phosphate- | tolyltrifluoro- | | naphthyl)(PCy₃)₂/ | carbonate | |
| | tris- | borate | | PCy₃ σ-complex | hydrate | |
| | trifluoroethyl | | | | | |
| | ester | | | | | |
| 2.24 | Tyr-O- | potassium p- | THF | Ni^{II}Cl(1- | potassium | 50° C. |
| | phosphate- | tolyltrifluoro- | | naphthyl)(PCy₃)₂/ | phosphate | |
| | tris- | borate | | PCy₃ σ-complex | (K₃PO₄) | |
| | trifluoroethyl | | | | hydrate | |
| | ester | | | | | |
| 2.25 | Tyr-O- | potassium p- | THF | Ni^{II}Cl(1- | diisopropyl | 50° C. |
| | phosphate- | tolyltrifluoro- | | naphthyl)(PCy₃)₂/ | ethylamine | |
| | tris- | borate | | PCy₃ σ-complex | (DIEA) | |
| | trifluoroethyl | | | | | |
| | ester | | | | | |
| 2.26 | Tyr-O- | potassium p- | THF | Ni^{II}Cl(1- | potassium | 50° C. |
| | phosphate- | tolyltrifluoro- | | naphthyl)(PCy₃)₂/ | hydroxide | |
| | tris- | borate | | PCy₃ σ-complex | | |
| | trifluoroethyl | | | | | |
| | ester | | | | | |

Sonagashira Reactions

| 3.1 3.2 | Tyr-O- phosphate Tyr-O- phosphate | 4-ethynyltoluene 4-ethynyltoluene | N,N-DMF N,N-DMF | Palladium(II) β- Oxoiminato- phosphane Complex Palladium(II) β- Oxoiminato- phosphane | potassium carbonate triethylamine | 50° C. 50° C. | 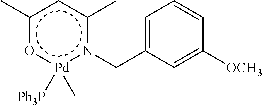 |
|---|---|---|---|---|---|---|---|
| | | | | Complex | | | |
| 3.3 | Tyr-O- | 4- | N,N-DMF | Palladium(II) β- | cesium | 50° C. | |
| | phosphate | ethynyltoluene | | Oxoiminato- | carbonate/ | | |
| | | | | phosphane | DBU | | |
| | | | | Complex | | | |
| 3.4 | Tyr-O- | 4- | N,N-DMF | Palladium(II) β- | piperidine | 50° C. | |
| | phosphate | ethynyltoluene | | Oxoiminato- | | | |
| | | | | phosphane | | | |
| | | | | Complex | | | |
| 3.5 | Tyr-O- | 4- | acetonitrile | PdCl₂(CH₃CN)₂/ | potassium | 50° C. | |
| | phosphate | ethynyltoluene | | Xphos | carbonate | | |
| 3.6 | Tyr-O- | 4- | acetonitrile | PdCl₂(CH₃CN)₂/ | triethylamine | 50° C. | |
| | phosphate | ethynyltoluene | | Xphos | | | |
| 3.7 | Tyr-O- | 4- | acetonitrile | PdCl₂(CH₃CN)₂/ | cesium | 50° C. | |
| | phosphate | ethynyltoluene | | Xphos | carbonate/ | | |
| | | | | | DBU | | |
| 3.8 | Tyr-O- | 4- | acetonitrile | PdCl₂(CH₃CN)₂/ | piperidine | 50° C. | |
| | phosphate | ethynyltoluene | | Xphos | | | |
| 3.9 | Tyr-O- | 4- | N,N-DMF | PdCl₂(PPh₃)₂/ | potassium | 50° C. | |
| | phosphate | ethynyltoluene | | AuCl(PPh₃) | carbonate | | |
| 3.10 | Tyr-O- | 4- | N,N-DMF | PdCl₂(PPh₃)₂/ | triethylamine | 50° C. | |
| | phosphate | ethynyltoluene | | AuCl(PPh₃) | | | |
| 3.11 | Tyr-O- | 4- | N,N-DMF | PdCl₂(PPh₃)₂/ | cesium | 50° C. | |
| | phosphate | ethynyltoluene | | AuCl(PPh₃) | carbonate/ | | |
| | | | | | DBU | | |
| 3.12 | Tyr-O- | 4- | N,N-DMF | PdCl₂(PPh₃)₂/ | piperidine | 50° C. | |
| | phosphate | ethynyltoluene | | AuCl(PPh₃) | | | |
| 3.13 | Tyr-O- | 4- | DMSO | PdCl₂(PCy₃)₂ | potassium | 50° C. | |
| | phosphate | ethynyltoluene | | | carbonate | | |
| 3.14 | Tyr-O- | 4- | DMSO | PdCl₂(PCy₃)₃ | triethylamine | 50° C. | |
| | phosphate | ethynyltoluene | | | | | |
| 3.15 | Tyr-O- | 4- | DMSO | PdCl₂(PCy₃)₄ | cesium | 50° C. | |
| | phosphate | ethynyltoluene | | | carbonate/ | | |
| | | | | | DBU | | |
| 3.16 | Tyr-O- | 4- | DMSO | PdCl₂(PCy₃)₅ | piperidine | 50° C. | |
| | phosphate | ethynyltoluene | | | | | |
| 3.17 3.18 | Tyr-O- phosphate Tyr-O- phosphate | 4- tolylpropargyl alcohol 4- tolylpropargyl alcohol | acetonitrile acetonitrile | Palladacycle 1- Xphos Palladacycle 1- Xphos | potassium carbonate triethylamine | 50° C. 50° C. | 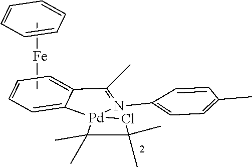 |
| 3.19 3.20 | Tyr-O- phosphate Tyr-O- phosphate | 4- tolylpropargyl alcohol 4- tolylpropargly alcohol | acetonitrile acetonitrile | Palladacycle 1- Xphos Palladacycle 1- Xphos | cesium carbonate/ DBU piperidine | 50° C. 50° C. | 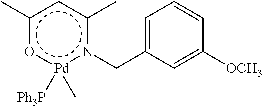 |
| 3.21 | Tyr-O- | 4- | N,N-DMF | Palladium(II) β- | potassium | 50° C. | |
| | phosphate- | ethynyltoluene | | Oxoiminato- | carbonate | | |
| | tris- | | | phosphane | | | |
| | trifluoroethyl | | | Complex | | | |
| | ester | | | | | | |
| 3.22 | Tyr-O- | 4- | N,N-DMF | Palladium(II) β- | triethylamine | 50° C. | |
| | phosphate- | ethynyltoluene | | Oxoiminato- | | | |
| | tris- | | | phosphane | | | |
| | trifluoroethyl | | | Complex | | | |
| | ester | | | | | | |
| 3.23 | Tyr-O- | 4- | N,N-DMF | Palladium(II) β- | cesium | 50° C. | |
| | phosphate- | ethynyltoluene | | Oxoiminato- | carbonate | | |
| | tris- | | | phosphane | DBU | | |
| | trifluoroethyl | | | Complex | | | |
| | ester | | | | | | |
| 3.24 | Tyr-O- | 4- | N,N-DMF | Palladium(II) β- | piperidine | 50° C. | |
| | phosphate- | ethynyltoluene | | Oxoiminato- | | | |
| | tris- | | | phosphane | | | |
| | trifluoroethyl | | | Complex | | | |
| | ester | | | | | | |
| 3.25 | Tyr-O- | 4- | acetonitrile | PdCl₂(CH₃CN)₂/ | potassium | 50° C. | |
| | phosphate- | ethynyltoluene | | Xphos | carbonate | | |
| | tris- | | | | | | |
| | trifluoroethyl | | | | | | |
| | ester | | | | | | |
| 3.26 | Tyr-O- | 4- | acetonitrile | PdCl₂(CH₃CN)₂/ | triethylamine | 50° C. | |
| | phosphate- | ethynyltoluene | | Xphos | | | |
| | trifluoroethyl | | | | | | |
| | ester | | | | | | |
| 3.27 | Tyr-O- | 4- | acetonitrile | PdCl₂(CH₃CN)₂/ | cesium | 50° C. | |
| | phosphate- | ethynyltoluene | | Xphos | carbonate/ | | |
| | tris- | | | | DBU | | |
| | trifluoroethyl | | | | | | |
| | ester | | | | | | |
| 3.28 | Tyr-O- | 4- | acetonitrile | PdCl₂(CH₃CN)₂/ | piperidine | 50° C. | |
| | phosphate- | ethynyltoluene | | Xphos | | | |
| | tris- | | | | | | |
| | trifluoroethyl | | | | | | |
| | ester | | | | | | |
| 3.29 | Tyr-O- | 4- | N,N-DMF | PdCl₂(PPh₃)₂/ | potassium | 50° C. | |
| | phosphate- | ethynyltoluene | | AuCl(PPh₃) | carbonate | | |
| | tris- | | | | | | |
| | trifluoroethyl | | | | | | |
| | ester | | | | | | |
| 3.30 | Tyr-O- | 4- | N,N-DMF | PdCl₂(PPh₃)₂/ | triethylamine | 50° C. | |
| | phosphate- | ethynyltoluene | | AuCl(PPh₃) | | | |
| | tris- | | | | | | |
| | trifluoroethyl | | | | | | |
| | ester | | | | | | |
| 3.31 | Tyr-O- | 4- | N,N-DMF | PdCl₂(PPh₃)₂/ | cesium | 50° C. | |
| | phosphate- | ethynyltoluene | | AuCl(PPh₃) | carbonate/ | | |
| | tris- | | | | DBU | | |
| | trifluoroethyl | | | | | | |
| | ester | | | | | | |
| 3.32 | Tyr-O- | 4- | N,N-DMF | PdCl₂(PPh₃)₂/ | piperidine | 50° C. | |
| | phosphate- | ethynyltoluene | | AuCl(PPh₃) | | | |
| | tris- | | | | | | |
| | trifluoroethyl | | | | | | |
| | ester | | | | | | |
| 3.33 | Tyr-O- | 4- | DMSO | PdCl₂(PCy₃)₂ | potassium | 50° C. | |
| | phosphate- | ethynyltoluene | | | carbonate | | |
| | tris- | | | | | | |
| | trifluoroethyl | | | | | | |
| | ester | | | | | | |
| 3.34 | Tyr-O- | 4- | DMSO | PdCl₂(PCy₃)₃ | triethylamine | 50° C. | |
| | phosphate- | ethynyltoluene | | | | | |
| | trifluoroethyl | | | | | | |
| | ester | | | | | | |
| 3.35 | Tyr-O- | 4- | DMSO | PdCl₂(PCy₃)₄ | cesium | 50° C. | |
| | phosphate- | ethynyltoluene | | | carbonate/ | | |
| | tris- | | | | DBU | | |
| | trifluoroethyl | | | | | | |
| | ester | | | | | | |
| 3.36 | Tyr-O- | 4- | DMSO | PdCl₂(PCy₃)₅ | piperidine | 50° C. | |
| | phosphate- | ethynyltoluene | | | | | |
| | tris- | | | | | | |
| | trifluoroethyl | | | | | | |
| | ester | | | | | | |

| | | | | | | | |
|---|---|---|---|---|---|---|---|
| 3.37 3.38 | Tyr-O- phosphate- tris- trifluoroethyl ester Tyr-O- phosphate- tris- trifluoroethyl ester | 4- tolylpropargyl alcohol 4- tolylpropargyl alcohol | acetonitrile acetonitrile | Palladcycle 1- Xphos Palladacycle 1- Xphos | potassium carbonate triethylamine | 50° C. 50° C. | 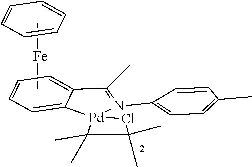 |
| 3.39 | Tyr-O- | 4- | acetonitrile | Palladacycle 1- | cesium | 50° C. | |
| | phosphate- | tolylpropargyl | | Xphos | carbonate/ | | |
| | tris- | alcohol | | | DBU | | |
| | trifluoroethyl | | | | | | |
| | ester | | | | | | |
| 3.40 | Tyr-O- | 4- | acetonitrile | Palladacycle 1- | piperidine | 50° C. | |
| | phosphate- | tolylpropargyl | | Xphos | | | |
| | tris- | alcohol | | | | | |
| | trifluoroethyl | | | | | | |
| | ester | | | | | | |

Peptides and Proteins

| | | |
|---|---|---|
| 4.2 | Tyr-O- | arylboronic |
| | phosphate | acid |
| | peptides | |
| 4.3 | Tyr-O- | arylboronic |
| | phosphate- | acid |
| | bis- | |
| | trifluoroethyl | |
| | ester | |
| | peptides | |
| 5.1 | Tyr-O- | 4- |
| | phosphate | ethynyltoluene |
| | peptides | or 4- |
| | | tolylpropargyl |
| | | alcohol |
| 5.2 | Tyr-O- | 4- |
| | phosphate- | ethynyltoluene |
| | bis- | or 4- |
| | trifluoroethyl | tolylpropargyl |
| | ester | alcohol |
| | peptides | |
| 6.1 | BSA-pTyr | arylboronic |
| | | acid |
| 6.2 | BSA-pTyr- | arylboronic |
| | O- | acid |
| | phosphate- | |
| | bis- | |
| | trifluoroethyl | |
| | ester | |
| | peptides | |
| 7.1 | BSA-pTyr | 4- |
| | | ethynyltoluene |
| | | or 4- |
| | | tolylpropargyl |
| | | alcohol |
| 7.2 | BSA-pTyr- | 4- |
| | O- | ethynyltoluene |
| | phosphate- | or 4- |
| | bis- | tolylpropargyl |
| | trifluoroethyl | alcohol |
| | ester | |
| | peptides | |

Biological Samples

| | | | | | | |
|---|---|---|---|---|---|---|
| 97 | Tyrosine- | TBD | TBD | TBD | TBD | TBD |
| | phosphorylated | | | | | |
| | whole | | | | | |
| | proteins | | | | | |
| | from culture | | | | | |
| 98 | Tyrosine- | TBD | TBD | TBD | TBD | TBD |
| | phosphorylated | | | | | |
| | whole | | | | | |
| | proteins | | | | | |
| | from culture | | | | | |
| 99 | Tyrosine- | TBD | TBD | TBD | TBD | TBD |
| | phosphorylated | | | | | |
| | whole | | | | | |
| | proteins | | | | | |
| | from culture | | | | | |
| 100 | Tyrosine | TBD | TBD | TBD | TBD | TBD |
| | phosphorylated | | | | | |
| | whole | | | | | |
| | proteins | | | | | |
| | from culture | | | | | |

What is claimed is:

1. A method of modifying a polypeptide comprising:
providing a polypeptide having at least one phosphorylated or sulfated tyrosine residue; and
forming a modified polypeptide by reacting the at least one phosphorylated or sulfated tyrosine residue with an organoboronic acid, a terminal alkyne, or a terminal alkene in the presence of an amount of a transition metal catalyst,
wherein the organoboronic acid, the terminal alkyne, or the terminal alkene is tethered to a structure,
wherein the transition metal catalyst contains Pd, Ni, Zn, Ir, Ru, Fe, Co, Cu, or Au, or a combination thereof.

2. The method of claim 1, including
forming a molecule of Formula (I): 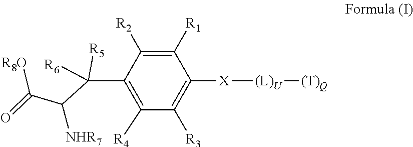
by reacting a molecule of Formula (II) or Formula (IV): 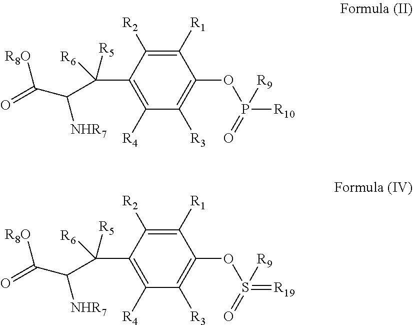
with a molecule of Formula (III):
Z-(L)_{U}-(T)_{Q}, Formula (III)
wherein
X is selected from the group consisting of 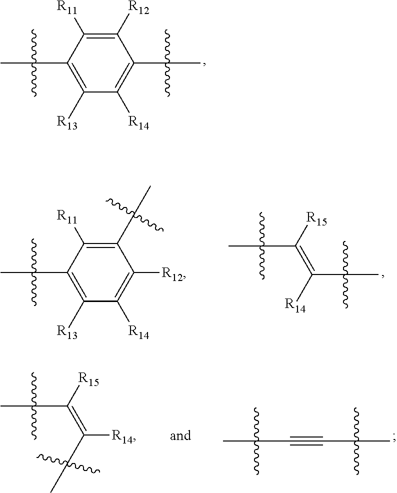
Z is selected from the group consisting of 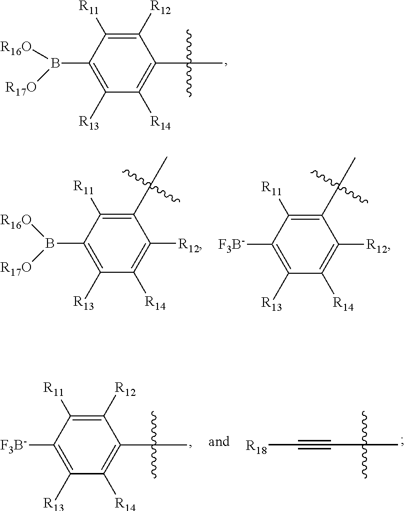
L is a linker;
U is an integer of 0 or 1;
T is the structure;
Q is an integer of 1, 2, or 3;
R₁, R₂, R₃, R₄, R₅, and R₆ are each independently selected from the group consisting of H, D, F, and C₁-C₈ alkyl;
R₇ and R₈ are each independently selected from the group consisting of H, D, C₁-C₈ alkyl and a polypeptide, wherein at least one of R₇ or R₈ is a polypeptide having from 2 to about 30,000 amino acids or wherein R₇ and R₈ combine to form a polypeptide having from 2 to about 30,000 amino acids;
R₉ and R₁₀ are each independently selected from the group consisting of OH, OD, ONa, OLi, OK, O—C₁-C₈ alkyl, NH—CF₃, and NH—CH₂—CF₃,
R₁₁, R₁₂, R₁₃, and R₁₄ are each independently selected from the group consisting of H, D, F, and C₁-C₈ alkyl;
R₁₅ is H or D;
R₁₆ and R₁₇ are each independently selected from the group consisting of H, D, C₁-C₈ alkyl, Ca, Mg, Na, Li, and K;
R₁₈ is H or D; and
R₁₉ is selected from the group consisting of OH, OD, ONa, OLi, OK, O—C₁-C₈ alkyl, NH—CF₃, and NH—CH₂—CF₃.

3. The method of claim 2, wherein the molecule of Formula (I) is selected from the group consisting of 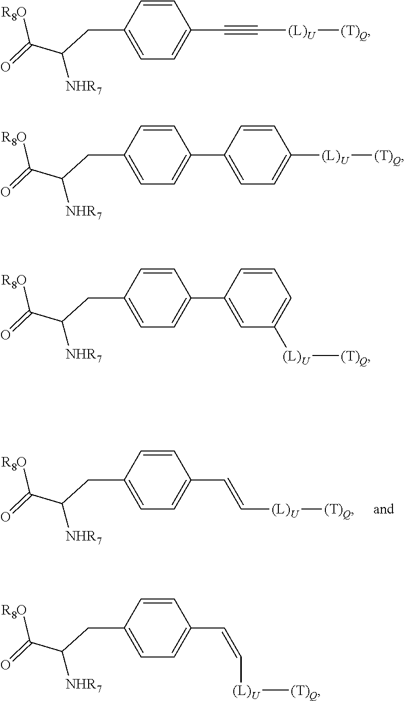
wherein R₇, R₈, L, U, T, and Q are as described in claim 2.

4. The method of claim 1, wherein the polypeptide is a protein.

5. The method of claim 2, wherein the R₇ and R₈ bridge to form a protein.

6. The method of claim 1, the modified polypeptide is formed by reacting the phosphorylated tyrosine residue with the organoboronic acid or the terminal alkyne.

7. The method of claim 1, wherein the modified polypeptide is formed by reacting the sulfated tyrosine residue with the organoboronic acid or the terminal alkyne.

8. The method of claim 2, including reacting the molecule of Formula (II) with the molecule of Formula (III).

9. The method of claim 2, including reacting the molecule of Formula (IV) with the molecule of Formula (III).

10. The method of claim 1, further comprising:
providing cells from a tissue or culture; and
disrupting the cells to provide the polypeptide having at least one phosphorylated or sulfated tyrosine residue.

11. The method of claim 1, wherein the transition metal catalyst includes a dialkyl biaryl-ligated palladium complex, a carbene-ligated palladium complex, [1,3-Bis(2,6-Diisopropylphenyl)imidazol-2-ylidene](3-chloropyridyl)palladium(II) dichloride; Dichloro[1,3-bis(2,6-Di-3-pentylphenyl)imidazol-2-ylidene](3-chloropyridyl)palladium(II); (1,3-Bis(2,6-diisopropylphenyl)imidazolidene) (3-chloropyridyl) palladium(II) dichloride; or a compound of Formula (IV) 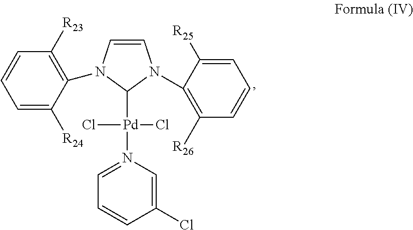
wherein R₂₃, R₂₄, R₂₅, and R₂₆ are each independently selected from a methyl group, an ethyl group, an isopropyl group, an isopentyl group, and an isoheptyl group, or a salt or mixture thereof.

12. The method of claim 1, wherein the transition metal catalyst includes palladium ligated to a ligand, wherein the ligand includes Sodium 2′-dicyclohexylphosphino-2,6-dimethoxy-1,1′-biphenyl-3-sulfonate hydrate; 2-Dicyclohexylphosphino-2′,6′-diisopropoxybiphenyl; 2-Dicyclohexylphosphino-2′,4′,6′-triisopropylbiphenyl; 2′-(Diphenylphosphino)-N,N′-dimethyl-(1,1′-biphenyl)-2-amine, 2-Diphenylphosphino-2′-(N,N-dimethylamino)biphenyl; 2-Dicyclohexylphosphino-2′-methylbiphenyl, 2-Methyl-2′-dicyclohexylphosphinobiphenyl; (2-Biphenyl)di-tert-butylphosphine; 2′-Dicyclohexylphosphino-2,4,6-trimethoxybiphenyl; 2′-Dicyclohexylphosphino-2-methoxy-1-phenylnaphthalene; 2-Dicyclohexylphosphino-2′-(N,N-dimethylamino)biphenyl; (2-Biphenyl)dicyclohexylphosphine, 2-(Dicyclohexylphosphino)biphenyl; 2-(Dicyclohexylphosphino)3,6-dimethoxy-2′,4′,6′-triisopropyl-1,1′-biphenyl; or a combination or mixture thereof.

13. The method of claim 1, wherein the structure includes a protein, a stable isotope label, biotin, streptavidin, a dendrimer, a fluorophore, or a radioactive label.

14. The method of claim 2, wherein the linker includes a polyethylene glycol segment or has a formula: —NH((CH₂)₂O)ₙ(CH₂)₂NH—, where n is an integer of from 0 to 50, or
wherein the dendrimer includes —(CH₂)₃— or —CH₂—CONH—CH₂— dendrons.

15. The method of claim 1, including
forming a molecule of Formula (I): 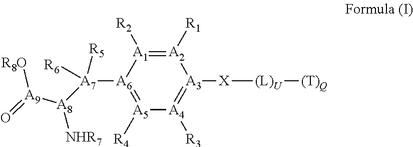
by reacting a molecule of Formula (II) or a molecule of Formula (IV): 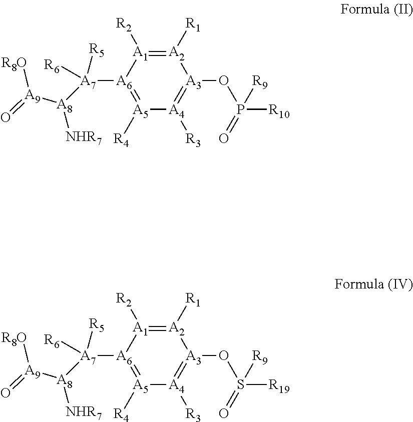
with a molecule of Formula (III):
Z-(L)_{U}-(T)_{Q}, Formula (III)
wherein
X is selected from the group consisting of 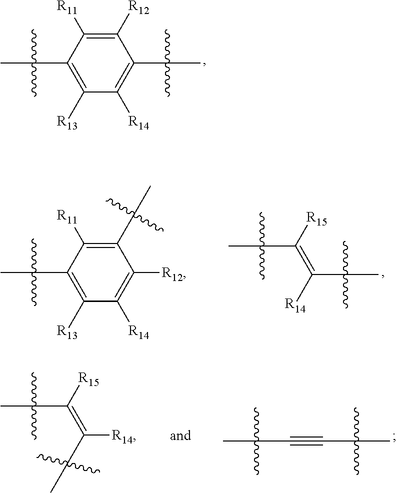
Z is selected from the group consisting of 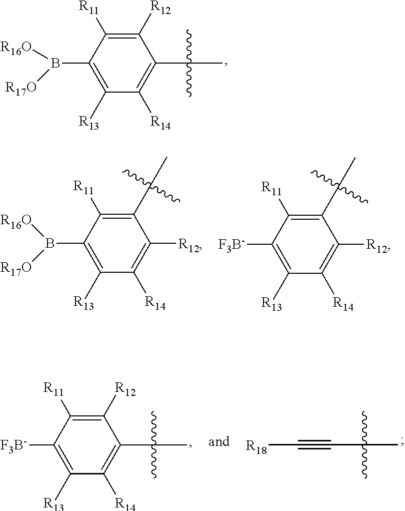
L is a linker;
U is an integer of 0 or 1;
T is the structure;
Q is an integer of 1, 2, or 3;
R₁, R₂, R₃, R₄, R₅, and R₆ are each independently selected from the group consisting of H, D, F, ¹²C₁-¹²C₈ alkyl, and ¹³C₁-¹³C₈;
R₇ and R₈ are each independently selected from the group consisting of H, D, ¹³C₁-¹³C₈, C₁-C₈ alkyl and a polypeptide, wherein at least one of R₇ or R₈ is a polypeptide having from 2 to about 30,000 amino acids or wherein R₇ and R₈ combine to form a polypeptide having from 2 to about 30,000 amino acids;
R₉ and R₁₀ are each independently selected from the group consisting of OH, OD, ONa, OLi, OK, O—C₁-C₈ alkyl, NH—CF₃, and NH—CH₂—CF₃,
R₁₁, R₁₂, R₁₃, and R₁₄ are each independently selected from the group consisting of H, D, F, and C₁-C₈ alkyl;
R₁₅ is H or D;
R₁₆ and R₁₇ are each independently selected from the group consisting of H, D, C₁-C₈ alkyl, Ca, Mg, Na, Li, and K;
R₁₈ is H or D,
R₁₉ is selected from the group consisting of OH, OD, ONa, OLi, OK, O—C₁-C₈ alkyl, NH—CF₃, and NH—CH₂—CF₃, and
A₁, A₂, A₃, A₄, A₅, A₆, A₇, A₈, and A₉ are each independently selected from the group consisting of ¹²C and ¹³C.
